(19) United States
(12) Patent Application Publication
(10) Pub. No.: US 2007/0207481 A1
(43) Pub. Date: Sep. 6, 2007

(54) Use of roma for characterizing genomic rearrangements

(76) Inventors: Michael Wigler, Cold Spring Harbor, NY (US); James Hicks, Cold Spring Harbor, NY (US); Larry Norton, New York, NY (US); Anders Zettenberg, Djursholm (SE)

Correspondence Address: FISH & NEAVE IP GROUP;ROPES & GRAY LLP, 1211 AVENUE OF THE AMERICAS, NEW YORK, NY, 10036-8704, US

(21) Appl. No.: 11/639,674

(22) Filed: Dec. 14, 2006

Related U.S. Application Data
(60) Provisional application No. 60/751,382, filed on Dec. 14, 2005.
(60) Provisional application No. 60/857,921, filed on Nov. 8, 2006.

(51) Int. Cl. C12Q 1/68; G06F 19/00; C07H 21/04

(52) U.S. Cl. 435/6; 702/20; 536/24.3

(57) ABSTRACT

The present invention relates to methods and compositions for detecting genomic rearrangements (e.g., amplification) at one or more genetic loci and various applications of such methods and compositions. Examples of genetic loci include HER2, TOP2A and other loci on the human chromosome 17.

### CROSS-REFERENCE TO RELATED APPLICATIONS

This application claims the benefit of and priority to U.S. Provisional Application No. 60/751,382, filed on Dec. 14, 2005, and No. 60/857,921, filed on Nov. 8, 2006, the contents of which are hereby incorporated by reference in their entirety.

### FIELD OF THE INVENTION

The present invention relates to methods and compositions for detecting genomic rearrangements (e.g., amplification) at one or more genetic loci and various applications of such methods and compositions.

### BACKGROUND OF THE INVENTION

Genomic rearrangements, including amplifications and deletions, account for the onset, development and progression of many diseases. Well-known examples include various cancers, and inherited disorders and predispositions. As each individual patient, as well as each individual tumor, has certain unique genetic traits, patients and tumors with similar phenotypic characteristics may not have the same underlying genotypes, and therefore, may respond differently to the same treatment.

A precise diagnosis is the first requirement for rational therapy. Cancer, a complex disease family that accounts for every fourth death in the United States, is no exception. Yet, the classical histopathological and clinical criteria used to assess the likelihood of response to the most commonly used modalities used to treat cancer and other diseases and disorders are inadequate predictors of treatment efficacy. Consequently, there is a significant and unmet need for accurate diagnostic methods that improve patient care and disease outcome.

Cancer is a genetic disease characterized by the progressive accumulation of lesions in the tumor genome. The number, severity and types of these lesions determine the biological properties of a given tumor. However, tools for high-resolution, comprehensive genome analysis have been lacking and consequently no cancer genome signatures that predict a patient's response to anti-cancer modalities have been discovered.

Pharmacogenetics and pharmacogenomics, the sciences that study the effects of genotype on individual drug responses in order to improve the safety and efficacy of drug therapy, were developed as a result of the recent sequencing of the human genome and other technological advances.

Despite these advances, there are few drugs or therapeutic regimens to date which have been successfully tailored for the individual patient or for a particular patient subpopulation (treatment stratification). One well-known example is the use of HER2 (ERBB-2) gene amplification or overexpression as a diagnostic tool for selecting breast cancer patients to be treated with Herceptin®. However, it is unclear whether the current technology has failed to detect certain genetic rearrangements at the HER2 locus, and therefore has excluded certain breast cancer patients who may respond to and benefit from Hercepting, alone or combined with another chemotherapy agent.

Further, based on existing diagnostic technologies, including fluorescence in situ hybridization, or “FISH,” 35-40% of breast cancer patients also receive a therapy (e.g., anthracycline) that targets a locus near the HER2 locus, topoisomerase 2A (TOP2A). It remains unclear, however, whether all these patients can benefit from such combination therapy.

Hence, a need exists for a robust technology, alone or in combination with other methods for genome profiling, that can determine more accurately the genetic determinants of a patient and/or a patient's tumor or other affected cells, that correlate with individual factors that may have led to the patient's phenotypic disease, and that point to features in the patient's genetic background that may lead to different responses to a given drug or combination therapy.

### SUMMARY OF THE INVENTION

The present invention provides methods and compositions that address the above discussed needs. The methods and compositions are particularly useful in detecting genomic rearrangements, such as amplification or deletion, or changes in copy number of any chromosomal region, especially at high resolutions of, e.g., 100, 60, 50, 35, 30, 25, 20, 15, 10 5 or 1 kilobase(s); or 800, 600, 400, 200, 100, 50 or fewer bases. In particular embodiments, the present invention provides methods and compositions that can be used to detect, distinguish and characterize at high resolution genomic rearrangements that may not be detectable by other methods currently employed to measure copy number of genomic regions, segments or loci, such as, for example, FISH.

In certain embodiments, the present invention also provides methods and compositions directed to assessing or predicting whether a patient is likely to respond to a particular drug or therapeutic regimen by analyzing that patient's genomic profile, for example, or a region of the patient's genomic profile that includes one or more genetic or genomic loci of interest. The methods and compositions of the invention are useful in determining a therapeutic regimen for an individual patient, the preferred therapy or therapeutic regimen being one that targets or treats one or more physiological pathways affected by the genetic rearrangements (e.g., one or more amplifications and/or deletions) identified in the patient's genomic profile, thereby ameliorating that patient's condition. The methods and compositions are useful in evaluating the suitability of a particular therapy or therapeutic regimen for a particular patient.

Certain embodiments of the present invention relate to methods for assessing the likelihood of a patient's response to a therapy that targets or treats one or more downstream effects of chromosomal rearrangement at a particular genetic locus X. For purposes of discussion, the genetic locus X (X) and a linked chromosomal region (R) are separated by interspersed region (I). The relative copy numbers of regions (R), (I) and (X) are referred to as (r), (i) and (x), respectively. The copy number of one or more segments of genomic DNA comprising the genetic locus (X) relative to that of a linked chromosomal region (R) is determined from DNA extracted from one or more diseased or affected cells of the patient, such as cancer or tumor cells or affected cells of an organ or tissue associated with a particular condition, disease or disorder. In particular embodiments, the copy number (i) of one or more segments of genomic DNA interspersed between the genetic locus X (X) and the linked chromosomal region (R) is determined relative to either or both the copy number of the linked region (r) and of the genetic locus X (x). “Linked” genetic loci refer to discrete segments of DNA that map to the same chromosome or chromosomal region. “Locus” (or “loci”) is not required to include the entire genomic sequence of any gene of interest; a genetic locus X, for example, a HER2 locus, includes any portion or portions of any size of the gene X's (e.g., the HER2 gene's) genomic sequence; a genetic locus X may also include any portion or portions of any size of two or more genes' genomic sequences. In particular embodiments, the linked chromosomal region includes a chromosomal centromere linked with a genetic locus X of interest. In other embodiments, the linked chromosomal region includes one or more loci, for example, one or more neighboring loci, of the genetic locus X.

The relative copy numbers of regions (R) and/or (I), and (X) may be used to deduce whether there have been one or more amplification and/or deletion events at or near the genetic locus X, which may be masking other rearrangement events at or encompassing genetic locus X. Accordingly, this is an especially useful method, for example, when one or more rearrangement events at the genetic locus X have occurred within a background of an earlier or a larger separate chromosomal rearrangement event, hence changing the relative copy number of sequences adjacent and/or distal to the genetic locus X.

Accordingly, relative copy numbers of regions (R) and/or (I), and (X) may be used to determine the likely response of the patient to a therapy that targets or treats an effect of the genetic rearrangements at the genetic locus X, such as misexpression (e.g., qualitative and/or quantitative changes in transcripts or transcript levels) of particular genes within the genetic locus X. Therapies directed to or especially effective in situations of over- or under-expression of the genetic locus X and/or its gene products may then be considered more likely to ameliorate or be effective in the patient's proposed treatment regimen, based on the relative copy number information that has been ascertained according to methods of the invention.

Thus, in certain embodiments where the copy number (i) of the interspersed region (I) is lower than that of both the region (R) and the genetic locus X (X), there is a certain likelihood that genetic locus X is within a genomic region that has undergone a deletion (hence lowering the relative copy number (i) of interspersed region (I)). The patient in this case may respond to a therapy targeting (e.g., ameliorating the effects of) amplification of the genetic locus X, especially when (X) and (R) are at about the same relative copy number, as (X) may be amplified within a region of chromosomal deletion, possibly as a result of selective pressure.

In certain other embodiments where the copy number (i) of the interspersed region (I) is higher than that of both the region (R) and the genetic locus X (X), there is a certain likelihood that genetic locus X is within a genomic region that has undergone an amplification (hence raising the relative copy number (i) of interspersed region (I)). The patient in this case may respond to a therapy targeting (e.g., ameliorating the effects of) deletion of the genetic locus X, especially when (X) and (R) are at about the same relative copy number, as (X) may be deleted within a region of chromosomal amplification, possibly as a result of selective pressure.

Certain embodiments of the invention provide a method for detecting a genomic or chromosomal rearrangement of a genetic locus X (X) in a patient. The method involves determining, in DNA extracted from one or more affected cells of the patient, the copy number of one or more segments of genomic DNA comprising the genetic locus X (X) relative to that of a linked chromosomal region (R), and in certain embodiments, to one or more segments of genomic DNA interspersed between genetic locus X and linked region (R). If the copy number (i) of the interspersed region (I) is different from that of the linked region (R) and/or of the genetic locus X (X), it may be deduced that there has been a chromosomal rearrangement (e.g., amplification or deletion) of the genetic locus X (X) in the affected cells relative to surrounding (adjacent or distal) sequences, segments or loci.

In certain embodiments, the genetic locus X is the HER-2 locus, and the linked region (R) is a region on chromosome 17, such as for example, the TOP2A locus or the RARA locus.

In certain particular embodiments of the invention, the genetic locus X is the TOP2A locus, and the linked region (R) is a region on chromosome 17, such as for example, the HER-2 locus or the RARA locus.

In other particular embodiments of the invention, one or more probes may be designed to target various locations within the interspersed region, which may be useful for any CGH experiment or for other methods for measuring copy number of specific genomic regions, such as for example FISH.

Kits with one or more compositions comprising at least one probe of the invention, a label and instructions for use are also provided.

### BRIEF DESCRIPTION OF THE FIGURES

FIG. 1 shows a segmented genomic profile of chromosome 17 in a breast tumor sample obtained by ROMA, which indicates amplification of the HER2 locus. The numbers on the X-axis refer to the probe numbers or positions in a 85K ROMA array. From the same sample, FISH has failed to detect the amplification, possibly as a result of employing a negative control probe that hybridizes to a region with the same relative copy number as HER2. Yet, HER2 is selectively amplified relative to the immediately adjacent loci, possibly as a result of selective pressure. A tumor with such a HER2 locus is likely to respond to HER2-targeted therapies.

FIG. 2 shows a segmented genomic profile of chromosome 17 in a breast tumor sample obtained by ROMA, which indicates amplification of the HER2 locus. The numbers on the X-axis refer to the probe numbers or positions in a 85K ROMA array. From the same sample, FISH has detected a very slight amplification.

FIG. 3 shows a segmented genomic profile of chromosome 17 in a breast tumor sample obtained by ROMA, which indicates amplification of the HER2 locus. The numbers on the X-axis refer to the probe numbers or positions in a 85K ROMA array. From the same sample, FISH has failed to detect the amplification.

FIG. 4 shows that ROMA is capable of discriminating between amplification of the HER2 locus and a linked proximal gene, TOP2A. The numbers on the X-axis refer to the probe numbers or positions in a 85K ROMA array. FIG. 4 also shows that ROMA can detect deletions of the BRCA1 gene on chromosome 17 in the same segmented genomic profile encompassing the HER2 and TOP2A loci, which may reduce the DNA repair capability of this region and contribute to further genomic rearrangement.

FIG. 5 shows the relative positions of various genes, including the HER2/ERBB2 and TOP2A loci, and other genome features (such as CpG islands) in the q12-q21.2 region of chromosome 17.

FIG. 6 shows a higher resolution of the 5′ portion of the HER2/ERBB2 locus on chromosome 17 and the chromosome band localized by FISH mapping clones. The vertical lines indicate the positions of the ROMA probes. The numbers (e.g., 35091588, 35100880, 35796237, 35800771) represent the respective chromosome positions on human chromosome 17 of certain ROMA probes used to analyze the HER-2 locus and its linked regions or loci.

FIG. 7 shows a higher resolution of the 3′ portion of the HER2/ERBB2 locus on chromosome 17 and the chromosome band localized by FISH mapping clones. The vertical lines indicate the positions of the ROMA probes. The numbers (e.g., 35091588, 35100880, 35796237, 35800771) represent the respective chromosome positions on human chromosome 17 of certain ROMA probes used to analyze the HER-2 locus and its linked regions or loci.

FIG. 8 shows a genomic profile obtained by ROMA with particular probes that can distinguish the copy number difference between the HER2/ERBB2 locus and the TOP2A locus. The numbers on the X-axis refer to the probe numbers or positions in a 390K ROMA array. The ROMA probes distinguished the copy number difference between these two loci: as indicated near position 312300 on the X-axis, the HER2 locus is amplified, whereas as indicated near position 312400 on the X-axis, the nearby TOP2A locus is not.

FIG. 9 shows a genomic profile obtained by ROMA with other probes that can distinguish the copy number difference between the HER2/ERBB2 locus and the TOP2A locus. The numbers on the X-axis refer to the probe numbers or positions in a 390K ROMA array. As shown in the figure, the ROMA probes distinguished the copy number difference between these two loci, although the HER2 amplicon nearly overlapped with the TOP2A locus.

FIG. 10 shows a genomic profile obtained by ROMA with other probes that can distinguish the copy number difference between the HER2/ERBB2 locus and the TOP2A locus. The numbers on the X-axis refer to the probe numbers or positions in a 390K ROMA array. As shown in the figure, the ROMA probes distinguished the copy number difference between these two loci.

FIGS. 11A and 11B show four different genomic profiles obtained by ROMA with other probes that can distinguish the copy number difference between the HER2/ERBB2 locus and the TOP2A locus. The numbers on the X-axis refer to the probe numbers or positions in a 390K ROMA array.

FIG. 12 shows the FISH results obtained by high-resolution probes designed to distinguish the TOP2A locus and a closely positioned locus, RARA.

FIG. 13 shows the ROMA profile of sample BTN48 at three different levels of magnification. Panel A depicts the entirety of chromosome 17 showing multiple peaks of amplification. Vertical (green and gray) reference lines indicate the limits of ERBB2 (left two) and TOP2A (right two) genes. Panel B is a partial enlargement of the 2 Mb region containing ERBB2. Panel C is a further enlargement showing the separation of the ERBB2 locus from the nearby amplicon (shoulder visible at far left).

### DETAILED DESCRIPTION OF THE INVENTION

The present invention generally relates to methods and compositions for detecting chromosomal rearrangements at any genetic locus (X) of interest. A chromosomal rearrangement can manifest itself in an increase in genomic copy number of a discrete genomic segment, an amplification; or a decrease in genomic copy number of a discrete genomic segment, a deletion. The present invention provides methods and compositions for assessing a patient's likely response to a particular therapy that targets or treats one or more effects of amplification or deletion of a genetic locus (X), especially in cases where (X) may be amplified within a region of chromosomal deletion, or where (X) may be deleted within a region of chromosomal amplification, either possibly as a result of selective pressure. In such cases, genomic rearrangement at genetic locus (X) may often be missed by state of the art diagnostic methods, and patient care and disease outcome are deleteriously affected.

The ability to detect genetic rearrangements of this sort depends on methods that can detect genomic rearrangements, such as amplification or deletion, or changes in copy number of any chromosomal region, at high resolutions of, e.g., 100, 60, 50, 35, 30, 25, 20, 15, 10, 5 and 1 kilobases; and 800, 600, 400, 200, 100, 50 or fewer bases. Accordingly, the present invention provides methods and compositions that can be used to detect genomic rearrangements that may not be detectable by other methods currently employed to measure copy number of genomic regions, segments or loci, such as, for example, fluorescence in situ hybridization, or “FISH.”

Particular embodiments of the present invention provide methods based on representational oligonucleotide microarray analysis (ROMA), and such methods are capable of detecting at high resolution certain chromosomal rearrangements that cannot be detected by lower resolution or more narrowly focused techniques, such as for example, by FISH. For example, if a chromosomal region has been subjected to one or more deletion events in the cancer cell, amplification of a genetic locus (X) near to or within that otherwise deleted region may be detected by FISH as having a normal copy number. Conversely, if a chromosomal region has been subjected to amplification, deletion of a genetic locus near that region may be detected by FISH as having a normal copy number. Accordingly, the present disclosure provides methods and compositions capable of detecting and identifying chromosomal rearrangements that other techniques fail to detect or tend to fail to detect (e.g., due to lower sensitivity, lower signal-to-noise ratio, narrower dynamic range, or lower resolution).

In certain embodiments, ROMA-based genomic profiling methods are used to identify particular chromosomal regions that exhibit rearrangements. After those regions are identified, probes may be designed that target these regions, which can then be used for verifying absolute copy number of segments or genetic loci near to or within these regions. Other techniques, such as FISH, can then employ the probes of the present disclosure for determining genomic copy number of a genetic locus of interest near the chromosomal regions targeted by the probes.

HER2

The present invention also provides a method for assessing a patient's (e.g., a cancer patient's) likely response to a HER2-based therapy, such as for example treatment with Herceptin®. In certain embodiments, the HER2-based therapy includes a combination therapy that includes one or more therapeutic agents targeting one or more other genes, such as, for example, TOP2A. For purposes of discussion, the HER2 locus (H) and a linked chromosomal region (R) are separated by interspersed region (I). The relative copy numbers of regions (R) (I) and (H) are referred to as (r), (i) and (h), respectively. Methods of this embodiment involve the step of measuring the relative copy number of one or more segments of genomic DNA comprising the HER2 locus (H) relative to that of a linked chromosomal region (R) present in DNA extracted from one or more diseased or affected cells of the patient, such as breast cancer cells. In particular embodiments, the relative copy number (i) of one or more segments of genomic DNA interspersed between the HER2 locus (H) and the linked region (R) is determined relative to the copy number of the linked region (R) and/or the HER2 locus (H). In particular embodiments, the linked region is a chromosomal centromere linked to a HER2 locus. In certain embodiments, the linked region includes the TOP2A locus. In certain embodiments, the linked region includes the RARA locus. In certain embodiments, the linked region includes one or more other loci on chromosome 17, in particular, q17-q21.2 of chromosome 17.

The relative copy numbers of regions (R) and/or (I), and (H) ((r) and/or (i), and (h)) may be used to deduce whether there have been one or more amplification and/or deletion events at or near the HER2 locus (H), which may be masking other rearrangement events at or encompassing the HER2 locus (H). Accordingly, this is an especially useful method, for example, when one or more rearrangement events at the HER2 locus (H) have occurred within a background of an earlier or a larger separate chromosomal rearrangement event, hence changing the relative copy number of sequences adjacent and/or distal to the HER2 locus (H).

Accordingly, relative copy numbers of regions (R) and/or (I), and (H) may be used to determine the likely response of the patient to a therapy that targets or treats an effect of the genetic rearrangements at the HER2 locus (H), such as misexpression or disruption of normal mRNA expression of HER2 and potentially other genes at or near the HER2 locus (H). Therapies directed to or especially effective in situations of over- or under-expression of the HER2 locus (H) and/or its gene products may then be considered more likely to be effective in the patient's proposed treatment regimen, based on the relative copy number information that has been ascertained according to methods of the invention.

Similarly, relative copy number of regions (R) that are near or overlap with the TOP2A locus may be used to determine the likely response of the patient to an adjuvant therapy that targets or treats an effect of genetic rearrangements at the TOP2A locus, such as amplification of the TOP2A locus.

Thus, in certain embodiments where the copy number of the interspersed region (i) is lower than that of both the region (R) and the HER2 locus (H), there is a certain likelihood that the HER2 locus (H) is within a genomic region that has undergone a deletion (hence lowering the relative copy number (i) of interspersed region (I)). The patient in this case may respond to a therapy targeting (i.e., ameliorating the effects of) amplification of the HER2 locus (H), especially when (H) and (R) are at about the same relative copy number, as (H) may be amplified within a region of chromosomal deletion, possibly as a result of selective pressure.

In certain other embodiments where the copy number of the interspersed region (i) is higher than that of both the region (R) and the HER2 locus (H), there is a certain likelihood that the HER2 locus (H) is within a genomic region that has undergone an amplification (hence raising the relative copy number (i) of interspersed region (I)). The patient in this case may respond to a therapy targeting (i.e., ameliorating the effects of) deletion of the HER2 locus (H), especially when (H) and (R) are at about the same relative copy number, as (H) may be deleted within a region of chromosomal amplification, possibly as a result of selective pressure.

The methods of the invention are especially useful, for example, when one or more rearrangement events at the HER2 locus (H) and/or one or more nearby loci such as the TOP2A locus have occurred within a background of an earlier or a larger separate chromosomal rearrangement event, hence changing the relative copy number of sequences adjacent and/or distal to the HER2 locus (H) and/or of sequences of one or more nearby loci. Therapies, known now and those developed in the future, directed to or especially effective in situations of over- or under-expression of the HER2 locus (H) and/or its gene products, and similarly, of the one or more nearby loci and/or their gene products, such as TOP2A, may then be considered more likely to ameliorate or be effective in the patient's proposed treatment regimen, once relative copy number information has been ascertained, preferably at high resolution.

Certain other embodiments of the present invention relate to a method for detecting one or more chromosomal rearrangements of a HER2 locus in a patient comprising determining the copy number of one or more segments of genomic DNA comprising a HER2 locus (H) relative to that of a linked chromosomal region (R) present in DNA extracted from one or more affected, e.g., cancer cells of the patient, such as breast cancer cells, and in certain embodiments, determining the copy number (i), relative to the copy number of the region (R) or the HER2 locus (H), of one or more segments of genomic DNA interspersed between the HER2 locus (H) and the linked chromosomal region (R). If the copy number of the interspersed region (i) differs from that of the region (R) and/or the HER2 locus (H), it may be deduced that there has been a chromosomal rearrangement (e.g., amplification or deletion) of the HER2 locus (H) in the affected cells of the patient relative to surrounding (adjacent or distal) sequences, segments or loci. In particular embodiments, the linked chromosomal region is a chromosomal centromere linked to a HER2 locus. In other embodiments, the linked chromosomal region is the TOP2A locus. In certain embodiments, the linked region includes the RARA locus. In certain embodiments, the linked region includes one or more other loci on chromosome 17, in particular, q17-q21.2 of chromosome 17.

In particular embodiments, the copy number (r) of the linked region (R) is higher than that of the interspersed region (I). In other particular embodiments, the copy number (r) of the linked region (R) is higher than that of each of the HER2 locus (H) and the interspersed region (I).

In particular embodiments, the copy number (r) of the linked region (R) is lower than that of the interspersed region (I). In other particular embodiments, the copy number (r) of the linked region (R) is lower than that of each of the HER2 locus (H) and the interspersed region (I).

In particular embodiments, the copy number (i) of the interspersed region (i) is lower than that of the linked region (R) and the HER2 locus (H). In other particular embodiments, the copy number (i) of the interspersed region (I) is higher than that of the linked region (R) and the HER2 locus (H).

In each of the above particular embodiments, the copy number (r) of the linked region (R) may be about the same as that of the HER2 locus (H).

Certain other embodiments of the present invention relate to a method for detecting one or more chromosomal rearrangements of a HER2 locus and one or more nearby loci including the TOP2A locus in a patient comprising determining the copy number of one or more segments of genomic DNA comprising a HER2 locus (H) relative to that of the one or more nearby loci present in DNA extracted from one or more cancer cells of the patient, such as breast cancer cells. In certain embodiments, the method can detect the difference in one or more chromosomal rearrangements, such as copy number difference, between the HER2 locus and the TOP2A locus, which chromosomal rearrangements are closely positioned such that traditional FISH probes cannot detect copy number differences.

TOP2A

The present invention also provides a method for assessing a patient's (e.g., a cancer patient's) likely response to a TOP2A-based therapy. It has been reported that genomic rearrangements (e.g., amplification) of the TOP2A locus may sensitize a patient to chemotherapy. One current therapy for patients that exhibit rearrangements (e.g., amplifications) at the TOP2A locus includes treatment with an anthracycline agent, typically in combination with one or more other chemotherapeutic agents. Anthracyclines include a class of chemotherapeutic agents based on daunosamine and tetra-hydro-naphthacene-dione. Examples of anthracycline agents include but are not limited to: daunorubicin; doxorubicin; epirubicin; idarubicin; mitoxantrone; and various dosage forms or formulations of these agents, such as liposomal formulations, including pegylated liposomal formulations, nanoparticles, other amphipathic vehicles and the like.

In certain embodiments, the TOP2A-based therapy includes a combination therapy that includes one or more therapeutic agents targeting one or more other genes, such as, for example, HER2. Similar to the HER2 discussion above, the TOP2A locus (T) and a linked chromosomal region (R) are separated by interspersed region (I). The relative copy numbers of regions (R) (I) and (T) may be referred to as (r), (i) and (t), respectively. Methods of this embodiment involve the step of measuring the relative copy number of one or more segments of genomic DNA comprising the TOP2A locus (T) relative to that of a linked chromosomal region (R) present in DNA extracted from one or more diseased or affected cells of the patient, such as breast cancer cells. In particular embodiments, the relative copy number (i) of one or more segments of genomic DNA interspersed between the TOP2A locus (T) and the linked region (R) is determined relative to the copy number (r) of the linked region (R) and/or the copy number (t) of the TOP2A locus (T). In particular embodiments, the linked region (R) is a chromosomal centromere linked to a TOP2A locus. In certain embodiments, the linked region includes the HER2 locus. In certain embodiments, the linked region includes the RARA locus. In certain embodiments, the linked region includes one or more other loci on chromosome 17, in particular, q17-q21.2 of chromosome 17.

The relative copy numbers of regions (R) and/or (I), and (T) may be used to deduce whether there have been one or more amplification and/or deletion events at or near the TOP2A locus (T), which may be masking other rearrangement events at or encompassing the TOP2A locus (T). Accordingly, this is an especially useful method, for example, when one or more rearrangement events at the TOP2A locus (T) have occurred within a background of an earlier or a larger separate chromosomal rearrangement event, hence changing the relative copy number of sequences adjacent and/or distal to the TOP2A locus (T).

Accordingly, relative copy numbers of regions (R) and/or (I), and (T) may be used to determine the likely response of the patient to a therapy that targets or treats an effect of the genetic rearrangements at the TOP2A locus (T), such as misexpression of TOP2A and potentially other genes at or near the TOP2A locus (T). Therapies directed to or especially effective in situations of over- or under-expression of the TOP2A locus (T) and/or its gene products may then be considered more likely to be effective in the patient's proposed treatment regimen, based on the relative copy number information that has been ascertained according to methods of the invention.

Similarly, relative copy number (r) of regions (R) that are near or overlap with the HER2 locus may be used to determine the likely response of the patient to a therapy that targets or treats an effect of genetic rearrangements at the HER2 locus, such as overexpression of HER2. One current therapy for patients that exhibit rearrangements (e.g., amplifications) at the HER2 locus includes treatment with Herceptin®.

Thus, in certain embodiments where the copy number (i) of the interspersed region (I) is lower than that of both the region (R) and the TOP2A locus (T), there is a certain likelihood that the TOP2A locus (T) is within a genomic region that has undergone a deletion (hence lowering the relative copy number (i) of interspersed region (I)). The patient in this case may respond to a therapy targeting (i.e., ameliorating the effects of) amplification of the TOP2A locus (T), especially when (T) and (R) are at about the same relative copy number, as (T) may be amplified within a region of chromosomal deletion, possibly as a result of selective pressure.

In certain other embodiments where the copy number (i) of the interspersed region (I) is higher than that of both the region (R) and the TOP2A locus (T), there is a certain likelihood that the TOP2A locus (T) is within a genomic region that has undergone an amplification (hence raising the relative copy number (i) of interspersed region (I)). The patient in this case may respond to a therapy targeting (i.e., ameliorating the effects of) deletion of the TOP2A locus (T), especially when (T) and (R) are at about the same relative copy number, as (T) may be deleted within a region of chromosomal amplification, possibly as a result of selective pressure.

The methods of the invention are especially useful, for example, when one or more rearrangement events at the TOP2A locus (T) and/or one or more nearby loci such as the HER2 locus or the RARA locus have occurred within a background of an earlier or a larger separate chromosomal rearrangement event, hence changing the relative copy number of sequences adjacent and/or distal to the TOP2A locus (T) and/or of sequences of one or more nearby loci. Therapies, known now and those developed in the future, directed to or especially effective in situations of over- or under-expression of the TOP2A locus (T) and/or its gene products, and similarly, of the one or more nearby loci and/or their gene products, such as HER2, may then be considered more likely to ameliorate or be effective in the patient's proposed treatment regimen, once relative copy number information has been ascertained, preferably at high resolution.

Certain other embodiments of the present invention relate to a method for detecting one or more chromosomal rearrangements of a TOP2A locus in a patient comprising determining the copy number of one or more segments of genomic DNA comprising a TOP2A locus (T) relative to that of a linked chromosomal region (R) present in DNA extracted from one or more affected, e.g., cancer cells of the patient, such as breast cancer cells, and in certain embodiments, determining the copy number (i) of an interspersed region (I), relative to the copy number of the region (R) or the TOP2A locus (T), of one or more segments of genomic DNA interspersed between the TOP2A locus (T) and the linked chromosomal region (R). If the copy number (i) of the interspersed region (I) differs from that of the region (R) and/or the TOP2A locus (T), it may be deduced that there has been a chromosomal rearrangement (e.g., amplification or deletion) of the TOP2A locus (T) in the affected cells of the patient relative to surrounding (adjacent or distal) sequences, segments or loci. In particular embodiments, the linked chromosomal region is a chromosomal centromere linked to a TOP2A locus. In other embodiments, the linked chromosomal region includes the HER2 locus, the RARA locus, and/or one ore more other loci on chromosome 17, in particular, in q17-q21.2 of chromosome 17.

In particular embodiments, the copy number (r) of the linked region (R) is higher than that of the interspersed region (I). In other particular embodiments, the copy number (r) of the linked region (R) is higher than that of each of the TOP2A locus (T) and the interspersed region (I).

In particular embodiments, the copy number (r) of the linked region (R) is lower than that of the interspersed region (I). In other particular embodiments, the copy number (r) of the linked region (R) is lower than that of each of the TOP2A locus (T) and the interspersed region (I).

In particular embodiments, the copy number (i) of the interspersed region (I) is lower than that of the linked region (R) and the TOP2A locus (T). In other particular embodiments, the copy number (i) of the interspersed region (I) is higher than that of the linked region (R) and the TOP2A locus (T).

In each of the above particular embodiments, the copy number (r) of the linked region (R) may be about the same as that of the TOP2A locus (T).

Certain other embodiments of the present invention relate to a method for detecting one or more chromosomal rearrangements of a TOP2A locus and one or more nearby loci including the HER2 locus in a patient comprising determining the copy number of one or more segments of genomic DNA comprising a TOP2A locus (T) relative to that of the one or more nearby loci present in DNA extracted from one or more cancer cells of the patient, such as breast cancer cells. In certain embodiments, the method can detect the difference in one or more chromosomal rearrangements, such as copy number difference, between the HER2 locus and the TOP2A locus, which chromosomal rearrangements are closely positioned such that traditional FISH probes cannot detect copy number differences.

ROMA vs. FISH

The present invention is based, in part, on a study that compares results of chromosomal rearrangements of the HER2 locus as detected by ROMA and FISH. ROMA is an ultra high resolution microarray-based Comparative Genomic Hybridization (CGH) tool that evolved from a technique termed RDA or representational difference analysis. See, e.g., Lucito et al. 2003 Genome Research 13:2291-2305; WO99/23256; U.S. Patent Application Publications Nos. 20040137473, 20050196799, and 20050266444. Like RDA, ROMA is capable of detecting differences present in different genomes, for example, between cancer genomes present in different cancer cells in the same or different patients, or between cancer genomes and normal genomes. Thus, ROMA has applications in the detection of genetic variation, in or between individuals, caused by deletions or duplications/ amplifications of genomic DNA involving one or more genetic or genomic loci, which may be related to progression and prognosis of cancer or other inherited or somatic diseases. RDA compares two genomes by subtractive hybridization, and ROMA is a high-throughput method which employs microarray analysis and also compares two genomes by subtractive hybridization.

ROMA employs oligonucleotide probes that are representations of a genome, made by, for example, restriction enzyme cleavage of the genomic DNA, and the oligonucleotide probes can be designed in silico. An exemplary enzyme is BglII, of which the cleavage sites are relatively uniformly distributed in the human genome. Digestion of DNA with BglII can create about 200,000 representational fragments of the human genome which are generally shorter than 1.2 kilobases, with an average spacing of about 17 kilobases. The representational oligonucleotide probes can be photoprinted onto microarray slides and then subjected to hybridization to genomic DNA of interest. Statistical data generated by the microarray hybridization can then be subjected to analysis by various algorithms, including, but not limited to, the circular binary segmentation that parses the probe ratio data into segments and creates a segmented genomic profile. See, e.g., Lucito et al., 2003.

FISH generally uses fluorescently labeled DNA molecules, i.e., probes, to analyze a genomic locus or a portion of genomic DNA of interest. The advantage of FISH is its high sensitivity at a single cell level. However, the technique is limited to the identity of the genomic loci of interest, that is, FISH can only detect genomic alterations at sites of the genome with known identity, i.e., nucleotide sequence, because, to achieve the high sensitivity, the nucleic acid sequences of FISH probes are pre-determined based on the genomic sites of interest. Currently, three types of FISH probes have been used for various genomic analyses: 1) locus specific probes that hybridize to a particular region of a chromosome and can generate data at single-cell level; 2) alphoid or centromeric repeat probes that are generated from repetitive sequences found at the centromeres of chromosomes and can be used to determine copy number of chromosomes but not of specific genetic loci; and 3) whole chromosome probes which are a plurality of smaller probes hybridizing to different sequences along the length of a chromosome and can be used to generate a spectral karyotype of a chromosome and to detect abnormality at the whole chromosome level, but not at specific genomic loci.

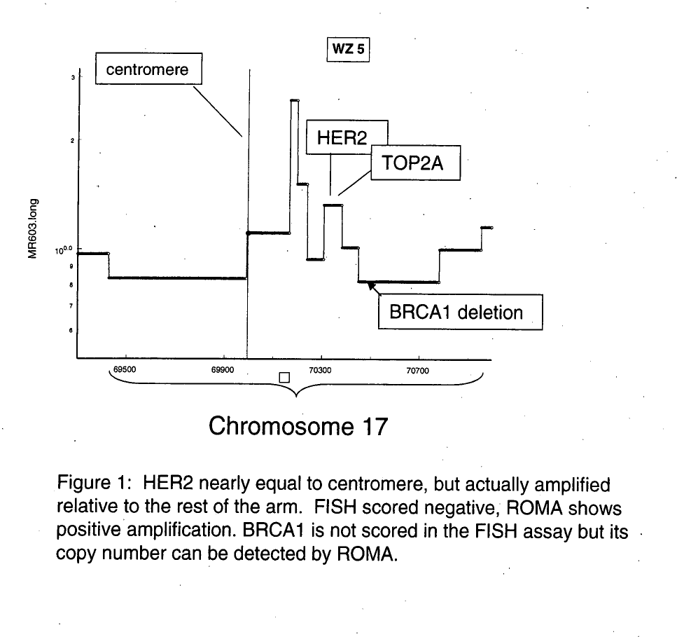

Accordingly, the present invention is based, in part, on studies that combined FISH analysis of specific, known genomic sites with ROMA. Such combination is capable of surveying an entire genome for chromosomal rearrangements, including copy number alterations at a high resolution and output. As shown in FIGS. 1-3, ROMA has unexpectedly detected chromosomal rearrangements at a HER2 locus, for which FISH has failed or nearly failed to do so.

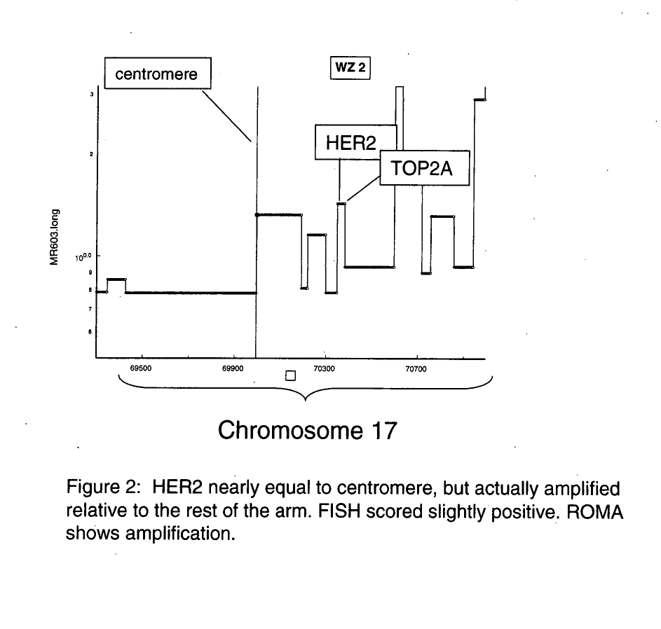

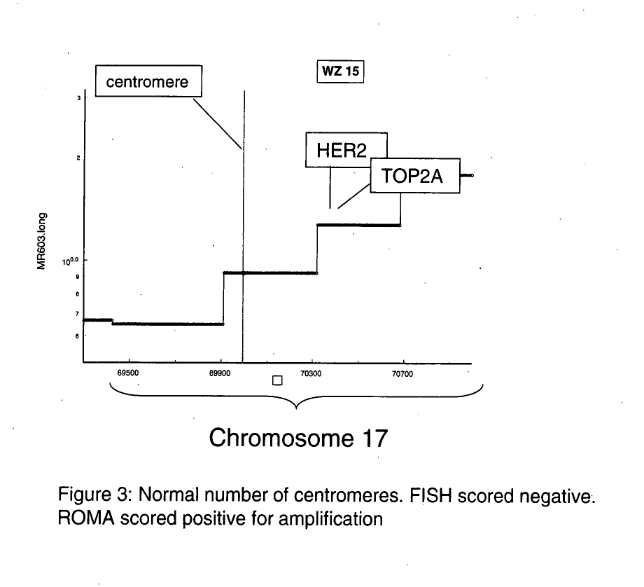

The ROMA technique has several novel uses and applications that are distinct improvements over current methods of FISH and ImmunoHistoChemistry (IHC). FIGS. 1-4 are magnifications of single chromosomes that are taken from the whole genome profile that results from each ROMA diagnostic test. FIG. 1 shows various examples of chromosome copy number alterations characterized by the selected amplification of the HER2 locus relative to neighboring sequences, but not relative to the centromere, the normalization point for FISH. The baseline or expected euploid copy number for chromosome 17 and its centromere is represented by the 10 0.0 (or 1) point on the Y axis. FIG. 1 shows both the HER2 and the topoisomerase 2A (TOP2A) loci in an amplicon of significant size relative to the deleted sequences on either side (in other words, both loci have been subjected to amplification), however it is only modestly higher than the copy number of the linked centromere. In contrast, the FISH score for this sample was negative, i.e., showing no amplification. FIGS. 2 and 3 illustrate other examples where ROMA indicates amplification relative to neighboring sequences and where the FISH score was negative or only slightly positive.

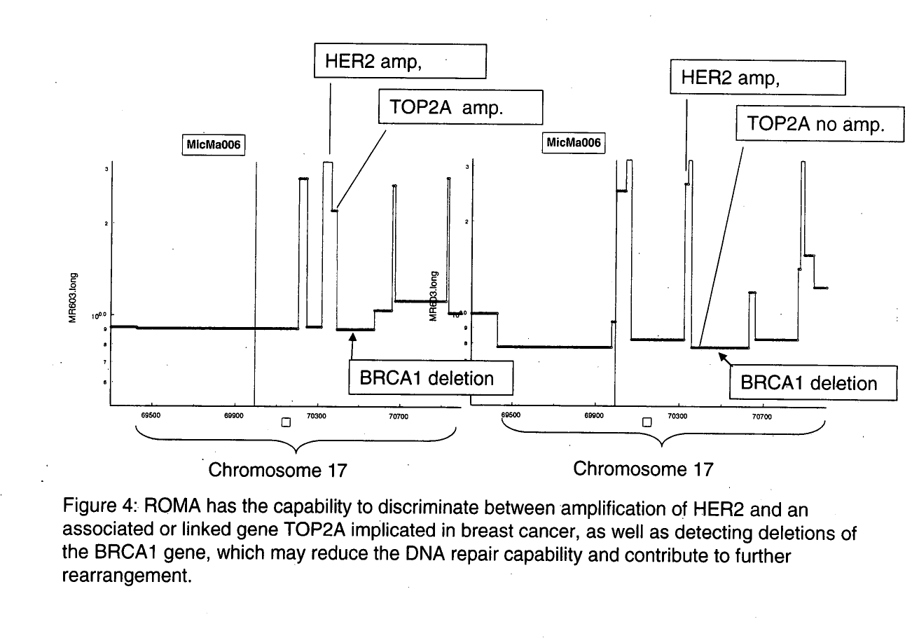

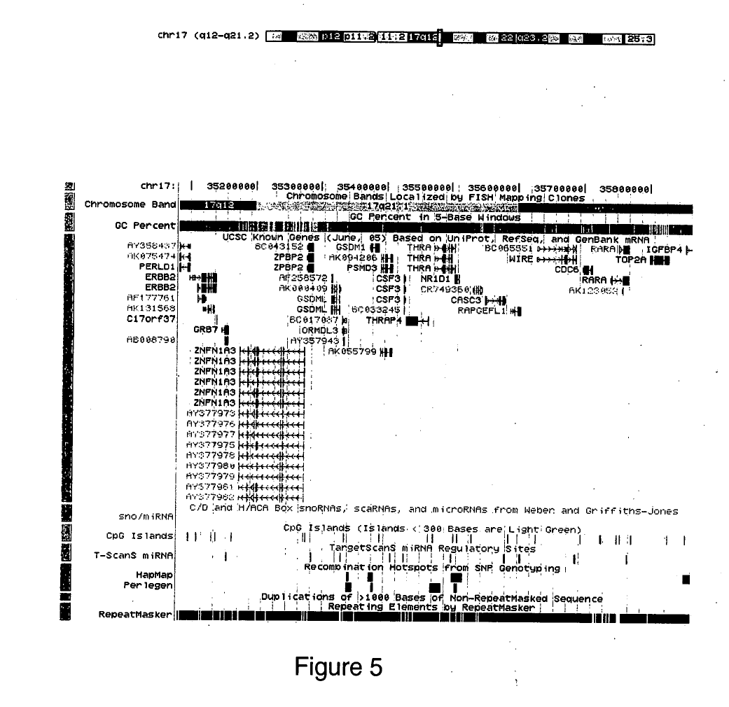

Another advantage of the ROMA method is that it can, in certain cases, separately profile genomic rearrangements at closely linked genetic loci, simultaneously and in a single experiment. This is in contrast to FISH, for example, where separate FISH probes must be used in different samples to measure copy number at two linked loci, even if they are closely linked. “Linked” genetic loci refer to discrete segments of DNA that map to the same chromosome, and often to the same or neighboring regions of the same chromosome. One skilled in the art will recognize that certain pathological conditions link DNA segments that, in normal humans, belong to different chromosomes. When such pathological conditions are studied, they can also be considered linked. Thus, ROMA allows the simultaneous independent measurement of HER2 and TOP2A, a closely linked gene that has also been implicated in breast cancer (Barghava, R, et al Am. J. Clin. Pathol. 2005. 123(6): 889-95), relative to their surrounding sequences. Specifically, the two profiles included in FIG. 4 show that a single ROMA test can distinguish whether the TOP2A locus is amplified or not independently of the HER2 locus. The test also distinguishes the deletion of the breast cancer susceptibility gene BRCA1, as shown. It is envisioned that ROMA will be similarly useful in quickly and efficiently, in a single test, distinguishing genomic rearrangements at other linked genetic loci involved in the etiology of disease.

In all three profiles shown in FIGS. 1-3, the HER2 locus has been amplified. However, FISH failed to detect the amplification in two out of the three samples and only scored slightly positive for HER2 amplification in the third sample. Had patients with these tumors been diagnosed using other methods, their HER2 amplifications would likely have been missed and their clinical situation misdiagnosed. Accordingly, the genomic profiles obtained by ROMA are useful in identifying certain chromosomal rearrangements that cannot be detected by other methods, such as FISH. This advantage of ROMA is attributable, at least in part, to the fact that ROMA can be used to determine the high resolution copy number of one or more chromosomal regions near or linked to a genetic locus of interest.

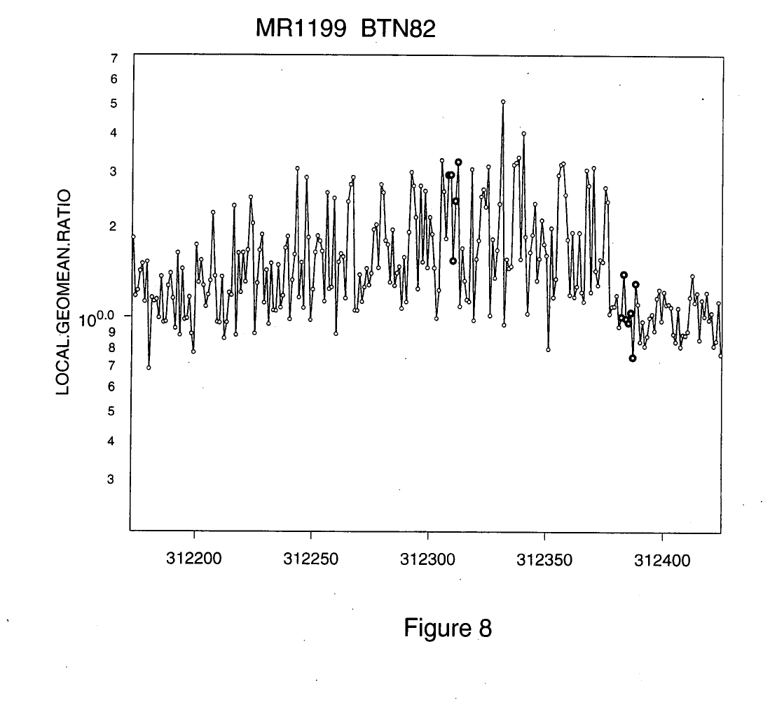

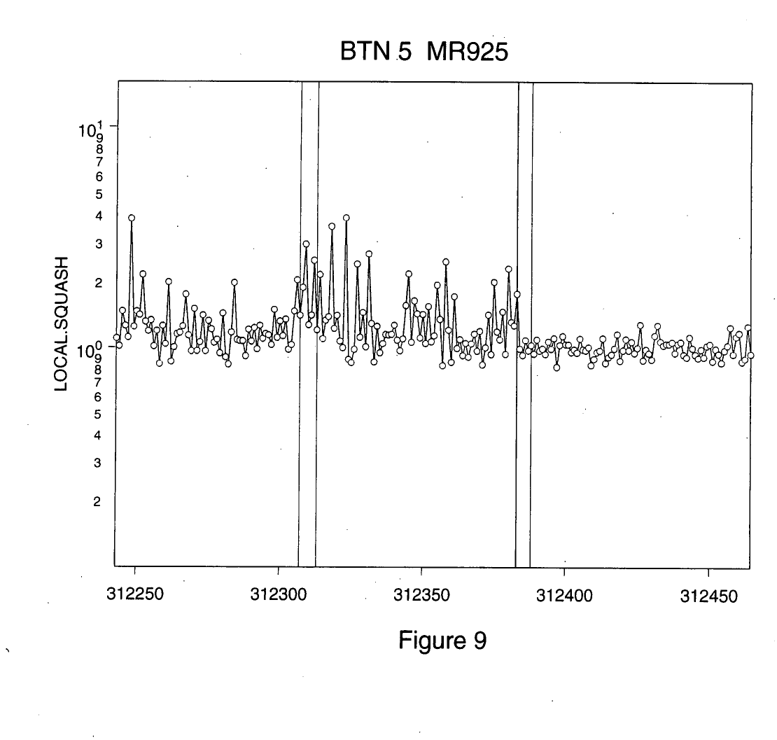

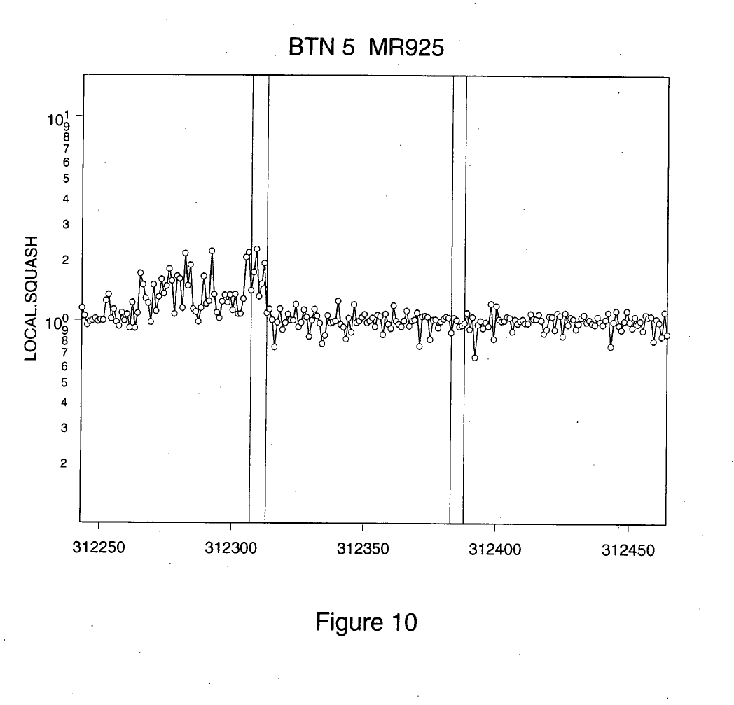

As shown in FIGS. 8-11A and B, ROMA allowed for the detection of copy number differences between the HER2 locus and TOP2A locus, where the traditional FISH probes failed to do so. Accordingly, the present invention provides methods for identifying differences in relative copy numbers between closely linked chromosomal loci (e.g., those which cannot be separately distinguished using FISH) using high resolution probes and methods disclosed herein. Methods for identifying probes that are capable of giving accurate, independent copy number readouts for linked chromosomal loci, and the probes identified by such methods, including but not limited to those disclosed herein, are provided by the present invention (see below).

Exemplary Application of the Invention Based on Chromosomal Rearrangements Detected at the HER2 Locus

The present invention provides a method of assessing a cancer patient's likely response to a HER2-based therapy involving characterizing segment copy number and chromosomal rearrangement(s) at a HER2 locus of segmented genomic DNA obtained from the patient's cancer cells. The disclosure contemplates various HER2-based therapies, and provides what is intended to be non-limiting examples, as follows.

The term “HER2-based therapy” as used herein refers to any therapy that targets the HER2 gene or HER2 protein, which results in reduced biological activity of either or both the gene and the protein. The therapy may inhibit HER2 gene expression transcriptionally, translationally, and/or post-translationally. The therapy may target and thus stimulate chromosomal rearrangements at or near a HER2 locus, which reduces HER2 gene or protein expression to a level insufficient to be oncogenic in an individual patient. The therapy may target the HER2 receptor, its ligand(s), or one or more other components of the HER2-mediated signal transduction pathway, and thereby inhibiting a biological activity of the HER2 protein. The therapy may involve one or more drugs, biologics, devices, homeopathic methods or products, or any combination thereof. For example, U.S. Patent Application Publication No. 20030171278 also discloses peptides and peptide derivatives (peptidomimetics or peptide analogs) that bind to the HER2 protein and inhibit its function. A HER2-based therapy may involve one or more HER2 inhibitors, and such inhibitors include, without limitation, peptides, peptide derivatives and analogs, non-peptide small molecules, antibodies, antibody portions or fragments, aptamers, antisense molecules, oligonucleotide decoys and nucleic acid molecules that mediate RNA interference (RNAi), such as, e.g., siRNAs, shRNAs, microRNAs and the like.

In certain embodiments, HER2-based therapy involves the use of biologics, such as for example, Herceptin®. Herceptin® is a humanized monoclonal antibody that specifically binds to an extracellular domain of the HER2 protein and approved by the U.S. Food and Drug Administration as a biologic for treating certain breast cancers. According to its Prescribing Information, “Herceptin® (Trastuzumab) as a single agent is indicated for the treatment of patients with metastatic breast cancer whose tumors overexpress the HER2 protein and who have received one or more chemotherapy regimens for their metastatic disease. Herceptin® in combination with paclitaxel is indicated for treatment of patients with metastatic breast cancer whose tumors overexpress the HER2 protein and who have not received chemotherapy for their metastatic disease. Herceptin® should be used in patients whose tumors have been evaluated with an assay validated to predict HER2 protein overexpression.”

In other embodiments, HER2-based therapy may involve small molecule drugs (SMDs), such as, for example, Lapatinib® (a potent, reversible inhibitor of both Her1 and HER2), that are engineered to inhibit the kinase activity of HER2. Any SMD or other agent that ameliorates one or more downstream effects of chromosomal rearrangement at the HER2 locus as detected by the methods of the invention are envisioned as being useful.

Exemplary Application of the Invention Based on Chromosomal Rearrangements Detected at the TOP2A Locus

The high-resolution detection by ROMA of chromosomal rearrangements at the TOP2A locus can also be used to determine the likely response of the patient to a therapy that targets or treats an effect of the genetic rearrangements at the TOP2A locus, such as inhibited expression or overexpression of TOP2A. The term “TOP2A-based therapy” as used herein refers to any therapy that targets the TOP2A gene or TOP2A protein, the overexpression of which has been linked to heightened sensitivity to certain chemotherapy. The therapy may enhance TOP2A gene expression transcriptionally, translationally, and/or post-translationally. The therapy may target and thus stimulate chromosomal rearrangements at or near a TOP2A locus, which leads to TOP2A gene or protein expression to a level sufficient to increase an individual patient's response or sensitivity to one or more chemotherapy agents. The therapy may involve one or more drugs, biologics, devices, homeopathic methods or products, or any combination thereof.

In certain embodiments, a therapy that targets TOP2A includes treatment with one or more anthracyclines. Anthracyclines include a class of chemotherapeutic agents based on daunosamine and tetra-hydro-naphthacene-dione. Examples of anthracycline agents include but are not limited to: daunorubicin; doxorubicin; epirubicin; idarubicin; mitoxantrone; and various dosage forms or formulations of these agents, such as liposomal formulations, including pegylated liposomal formulations, nanoparticles, other amphipathic vehicles and the like.

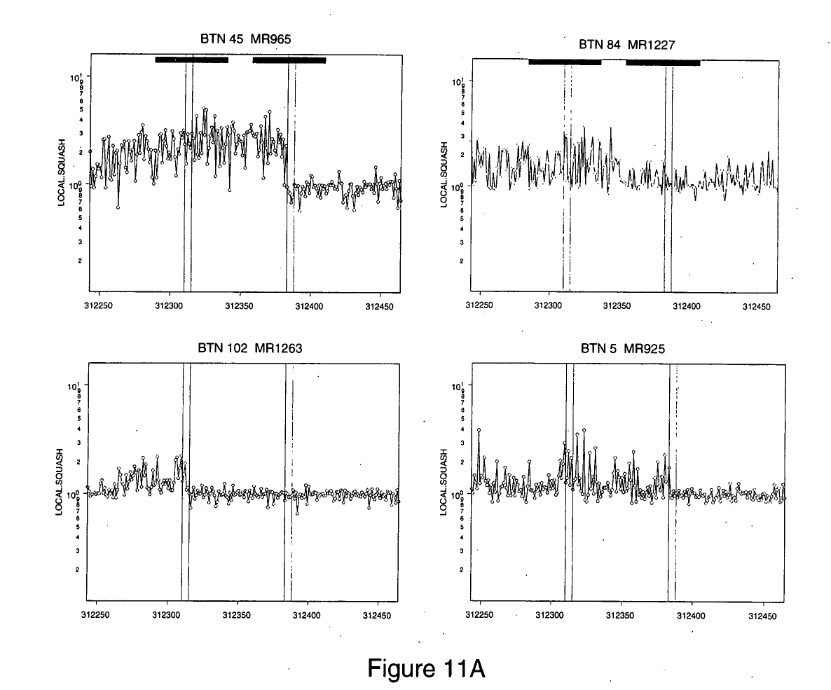

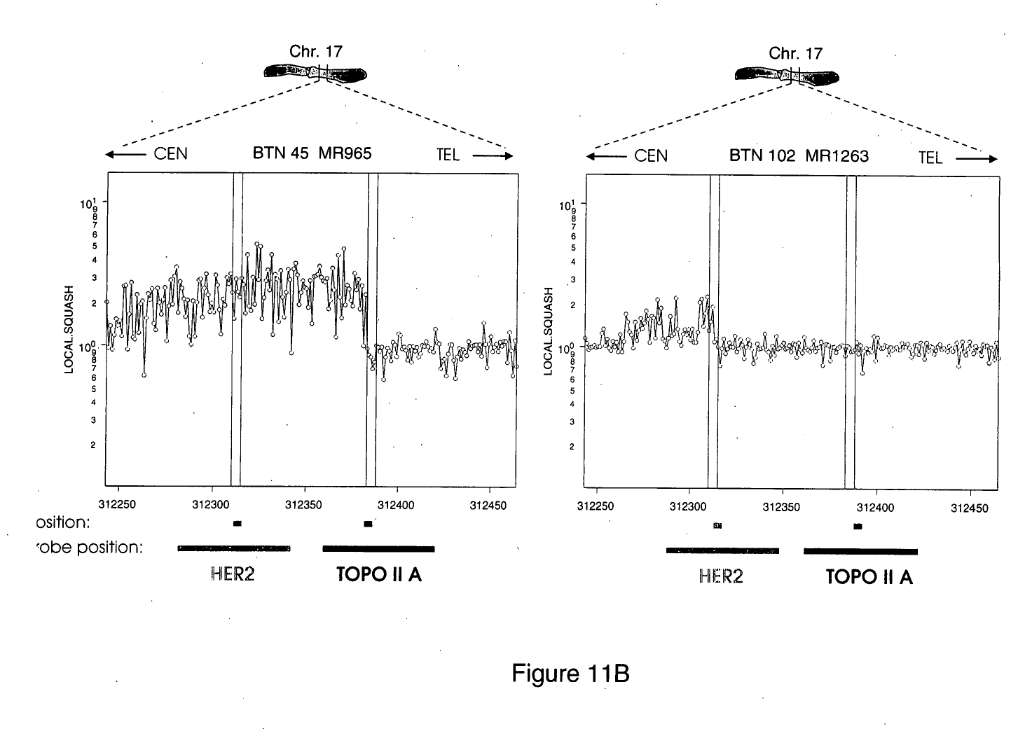

Co-amplification of the HER-2 and TOP2A genes has been linked to the effects of anthracyclines. Their role in predicting the outcome of anthracycline-based adjuvant chemotherapy for breast cancer patients has remained controversial. A recent study concludes: “Coamplification of HER-2/neu and TOP2A may define a subgroup of high-risk breast cancer patients who benefit from individually tailored and dose-escalated adjuvant anthracyclines.” Tanner et al., J Clin Oncol. 2006; 24: 2428-2436 (Epub date May 8, 2006). The same study also reports that “HER-2/neu amplification alone . . . was present in 32.7% of the tumors . . . TOP2A coamplification . . . was present in 37% of HER-2/neu-amplified tumors, [and] was associated with better relapse-free survival in patients treated with tailored and dose-escalated FEC (fluorouracil, epirubicin, and cyclophosphamide).” However, as exemplified in FIGS. 8-11A and 11B and discussed below, some of the patients receiving anthracycline-based adjuvant chemotherapy may not in fact have an amplified TOP2A locus, which may have led to the controversial results. That is because the conventional technology (e.g., FISH) could not distinguish certain copy number differences between closely linked chromosomal segments, such as for example, as shown in the genomic profile in FIG. 8 and the genomic profile on the left in FIG. 11B. As discussed next, it is estimated that only 12-15% of breast cancer patients with an amplified HER2 locus may in fact have an amplified TOP2A locus, and the remainder of those patients receiving anthracycline-based therapies may not have an amplified TOP2A locus. Thus, more than half of the patients currently receiving anthracycline-based therapies may not benefit from such therapies. In addition, anthracyclines are highly toxic compounds and can have significant adverse effects, such as cardiotoxicity, in patients. This mis-diagnosis may be detected by the methods and compositions of the present invention, as shown in the figures. Accordingly, the present invention provides new and improved methods and compositions that enable more accurate diagnoses and assessments of appropriate therapeutic regimens based on high resolution genomic profiling.

Linkage Between the HER2 Locus and the TOP2A Locus

It has been known for some time that in some cases where there is amplification of the HER2 or ERBB2 gene on chromosome 17q there are also amplifications of other genes flanking HER2. One such flanking gene, TOP2A is located approximately 700 kbp distal to HER2 on the same chromosome arm. Amplification of the TOP2A locus has been subjected to intense study because it has been proposed that elevated TOP2A levels will make tumors more susceptible to chemotherapy. It has been noted in the literature that this gene is not always co-amplified with HER2 and is considered to be a separate amplicon. Tanner et al., supra. Various studies have reported that co-amplification of TOP2A occurs in 30-35% of cases where HER2 is amplified. In clinical pathology practice, amplification of both the HER2 gene and the TOP2A gene are assayed by different versions of FISH. For each of the two FISH assays one or more commercial probes are available (e.g., DakoCytomation Her2 FISH pharmDx™). These commercial probes ostensibly measure the copy number (and thus the level of amplification) of the DNA encoding the target genes, in this case HER2 and TOP2A. Due to the limited sensitivity of the FISH procedure the probes are much longer (˜200 kbp) than the sequence of the genes themselves (˜35 kbp each) and may extend significant distances on either side of the gene that the FISH procedure ostensibly assays. Thus, the FISH procedure assays a region rather than a gene itself.

The present invention relates, in part, to the finding that the actual frequency of co-amplification of HER2 and TOP2A is less than 10%. This discrepancy as compared to previous studies is due to the vastly increased resolution of the breakpoint or edge of the HER2 amplicon, using a high resolution system, e.g., a ROMA microarray system comprising a 390 K microarray hybridization method that reports copy number for every 8 kbp of DNA sequence. By comparing the ROMA results to the results using the commercial FISH probes tested in the Memorial Sloan-Kettering Cancer Center pathology laboratory, it is estimated that the commercial FISH probes have mis-diagnosed TOP2A amplification in more than 50% of the FISH positive assays. Furthermore, in certain cases, the amplification breakpoint falls inside the TOP2A gene, leaving part of the gene un-amplified. This rearrangement may, in fact, ‘kill’ or silence the gene rather than amplifying it.

Accordingly, the methods and compositions of the present invention allow for 1) the determination and measurement of the exact breakpoints at the edges of the HER2 amplicons that have never been measured to this resolution in a statistically significant set of samples; 2) discovering that the existing FISH probes yield a large fraction of false positives, and 3) determination and measurement of certain breakpoints of HER2 amplicons within the TOP2A gene which have as yet unknown effects on the activity of the gene.

The methods and compositions described herein have many applications in medicine, especially oncology, as it is moving toward stratified and individualized treatments based on genomics and genetics. As noted above, Herceptin® is already being prescribed for patients with HER2 amplification, and TOP2A amplification has been cited as an indicator for a particular type of chemotherapy (adriamycin) when HER2 is amplified. The methods and compositions described herein enable accurate readouts for both genes separately. Methods for identifying and/or designing probes that are capable of giving accurate, independent copy number readouts for linked chromosomal loci, such as for HER2 and TOP2A—and the probes identified by such methods, including but not limited to those disclosed herein—are provided by the present invention.

Probes

The present invention also provides probes useful for detecting chromosomal rearrangements. In certain embodiments, a probe is provided for detecting a chromosomal rearrangement of a genetic locus X (e.g., a HER2 locus, a TOP2A locus, or a RARA locus) in a patient. The probe hybridizes to one or more genomic segments interspersed between the genetic locus X and a linked chromosomal region, and is capable of determining whether the relative copy number (i) of the interspersed region (I) is lower or higher than that of either or both of the linked region (R) and the genetic locus (X). In particular embodiments, a linked chromosomal region is a chromosomal centromere linked to the genetic locus X. In particular embodiments, a linked chromosomal region includes one or more genetic loci linked to the genetic locus X, such as for example the TOP2A locus and/or the RARA locus relative to the HER2 locus. Using endpoints such as those shown in the Figures and Tables herein (see, e.g., Tables 2 and 4 and Example 3), one or more probes are designed for a selected X, R, and I region based on knowledge of known boundaries or endpoints for loci of interest. In certain embodiments, the probes of the invention can be used in conventional FISH procedures which will enhance resolution of the FISH assays.

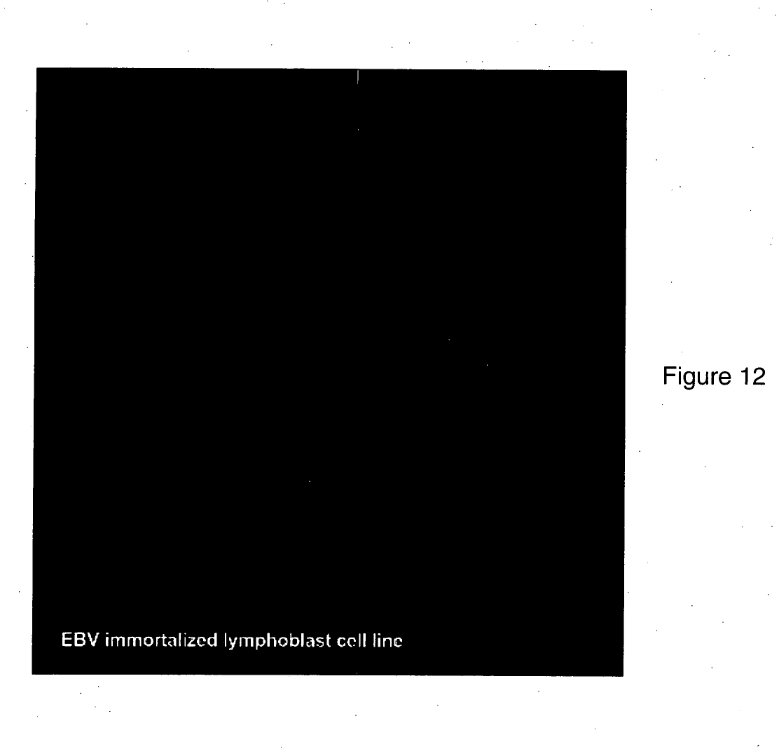

The probes of the present invention include a set of DNA sequences extracted from the human genome sequence that can be used to assay the copy number of DNA sequences in the genome of a patient's tumor/biopsy on chromosome 17 to a resolution of less than 50 kbp, optimally in the range of 1-10 kbp. The DNA sequences may be arrayed on a substrate for hybridization (microarray) of any of a variety of formats. Alternatively, a subset of these sequences, designed using a proprietary algorithm for selecting sequences for designing short FISH probes, has been created to design a set of FISH probes that can be used in standard pathology practice for accurately determining TOP2A copy number independent of HER2. An example of such probes used in a conventional FISH procedure is shown in FIG. 12.

Methods for Determining Copy Number

The present invention provides probes that can be employed in various methods capable of determining the copy number of a chromosomal region or locus and provides the following non-limiting examples.

DNA microarrays have been used to characterize alterations in genomic DNA copy number in cancer. Alterations in DNA copy number, including the large chromosomal gains and losses that characterize aneuploidy, as well as more localized regions of gene amplification and deletion, are a near-universal finding in human cancer. Mapping chromosomal regions of DNA amplification and deletion is useful in the localization of oncogenes and tumor suppressor genes, respectively. Alterations in DNA copy number have been mapped genome-wide using FISH-based techniques, including CGH and spectral karyotyping.

Laframboise et al., PLoS Comput. Biol. 2005 November;1(6):e65. Epub 2005 Nov. 25 reported a probe-level allele-specific quantitation procedure that extracts both copy number and allelotype information from single nucleotide polymorphism (SNP) array data to arrive at allele-specific copy number across the genome. The approach applies an expectation-maximization algorithm to a model derived from a novel classification of SNP array probes. MLPA (Multiplex Ligation-dependent Probe Amplification), MAPH (Multiplex Amplifiable Probe Hybridization) and SNP-typing, i.e., the Affymetrix 10K human SNP Array are also useful in detecting genomic copy number changes. See, e.g., Gert-Jan B. van Ommen et al. HGM2004 Poster Abstracts 3. Chip Technologies Poster 62.

Pollack et al., Nature Genetics 23, 41-46 (1999) discussed genome-wide analysis of DNA copy-number changes using cDNA microarrays. The use of cDNA microarrays for analysis of DNA copy-number variation offers some significant advantages over other array-based CGH methods which have relied on array targets comprised of large genomic DNA clones (for example BACs, or BAC-derived inter-Alu PCR products). High-density cDNA microarrays containing 10,000 genes or more are routinely employed for gene expression analyses, but no resource currently exists for full-genome coverage with large genomic clones. Another important advantage of cDNA microarray-based CGH is that DNA copy number and gene expression patterns can be characterized in parallel in the same sample. The ability to monitor gene amplification and expression in parallel and at high resolution may facilitate the identification of pathogenetically important genes in amplicons, and aid in the interpretation of the gene expression data being collected in studies of human tumors.

Herrick et al. taught an approach that was developed for the quantification of subtle gains and losses of genomic DNA (JCO, Vol. 97, Issue 1, 222-227, Jan. 4, 2000). The approach relies on a process called “molecular combing.” Molecular combing consists of the extension and alignment of purified molecules of genomic DNA on a glass coverslip. It has the advantage that a large number of genomes can be combed per coverslip, which allows for a statistically adequate number of measurements to be made on the combed DNA. Consequently, a high-resolution approach to mapping and quantifying genomic alterations is possible. The approach consists of applying fluorescence hybridization to the combed DNA by using probes to identify the amplified region. Measurements then are made on the linear hybridization signals to ascertain the region's exact size.

Other methods for detecting copy number alterations at specific loci are also available. Schaeffeler et al., Hum. Mutat. 2003 December;22(6):476-85, reported a TaqMan real-time PCR assay to specifically amplify genomic CYP2D6 by using a specific set of amplification primers and probe to effectively prevent amplification of CYP2D7 and CYP2D8 pseudogenes. The semiautomatic TaqMan assay allows high sample throughput and will be useful for pharmacogenetic studies and in the clinical setting.

Other Applications

Methods and compositions of the invention are useful in a number of applications including, but not intended to be limited to, those outlined below.

As described above, the present invention provides methods and compositions that make it feasible to detect and identify chromosomal rearrangements at or near one or more genetic loci of interest, which rearrangements are either not detectable or would likely score as negatives when only conventional methods, such as FISH, are used. Accordingly, the methods and compositions of the present invention make it possible to identify patients who are currently diagnosed as being unsuited or unresponsive to a particular treatment or therapy but who would, in fact, potentially respond to a particular therapy. Thus, an important application of the instant methods and compositions is in accurate and comprehensive diagnosis of patients and market expansion of known therapies.

For example, based on current diagnostic tools, Cognex® (tacrine hydrochloride for the treatment dementia of the Alzheimer's type) is deemed inefficacious in more than 50% of patients and Herceptin® is deemed inefficacious in more than 70% of patients. As demonstrated herein, certain amplification events at genomic loci, such as the HER2 locus, are not detected or are likely mis-classified as negative by current diagnostic tools such as those based on FISH analyses. Accordingly, diagnostic tools based on the instant methods and compositions are expected to enable more patients to be correctly diagnosed as those who will benefit from treatment with, and will facilitate an associated market expansion for, Herceptin® and other drugs whose efficacy is influenced by the copy number of one or more genetic loci.

Many other therapies have demonstrated varied efficacy in different patients due to genetic differences. For example, Evans et al., “Pharmacogenomics—Drug Disposition, Drug Targets, and Side Effects,” New England Journal of Medicine 348(6):538-549 (2003), discusses various therapies that show different responses in different patients. As its title suggests, the article discusses three categories of varied patient responses: 1) different patients metabolize or absorb the same drug differently due to genetic variations in drug-metabolizing enzymes (e.g., CYP3A family of P-450 enzymes) and drug transporters (e.g., MDR1); 2) different patients respond differently from the efficacy perspective due to genetic variations in drug targets (e.g., Table 1 of the article shows various genetic polymorphisms ion drug target genes that can influence drug response); and 3) different patients have different side effects resulting from the same therapy (e.g., Table 2 of the article shows the influence of genetic polymorphism in disease-modifying or treatment-modifying genes on drug effect or toxicity).

Accordingly, more accurate and comprehensive diagnosis of patients based on individual genomic profile information is useful in efficacy prediction, which can increase the power of clinical studies while decreasing the size of clinical trials. Diagnostic tools based on the instant methods and compositions can help stratify candidate patient populations for any given therapy, thereby optimizing clinical studies and outcome.

More accurate and comprehensive diagnosis of patients is also useful in identifying patients likely to experience common side effects due to toxicity and metabolic difficulties. The development of successful therapeutics has followed a well-established and costly evaluation process including phases I, II, and III clinical trials. Phase I studies aim to determine the maximally tolerated dose of the drug, its optimal schedule of administration and the dose-limiting toxicities. Historically, cytotoxic cancer therapies have been developed based on maximum tolerated doses (MTD), treating patients without understanding the tumor profile for likely responders. Hence, patients were often subjected to toxic therapies with limited therapeutic benefit. Further, current clinical trials typically do not account for patients′ individual traits (genetic, environmental, or other personal factors), and therefore often include patients who are not suitable candidates for the particular drugs or therapeutic regiments involved in the clinical trials. That over-inclusion without patient strata can seriously undercut efficacy or safety as demonstrated by a particular clinical trial, the cost of which can be significant. Accordingly, diagnostic tools based on the instant methods and compositions can help optimizing clinical studies by excluding patients who are at a higher risk or more likely to develop adverse responses to the test therapy, thereby making the clinical studies more efficient and cost-effective. More importantly, diagnostic tools based on the instant methods and compositions can help selecting and monitoring patients for receiving a marketed therapy, thereby reducing the incidence of adverse effects and potential withdrawal of the therapy from the market.

Accordingly, the present invention also provides kits, including diagnostic kits, which comprise one or more probes described or designed by the methods herein. A kit of the invention may further include a label. A kit of the invention may also include an instruction for use, e.g., in the form of an instruction manual.

The disclosure now being generally described, it will be more readily understood by reference to the following examples, which are included merely for purposes of illustration of certain aspects and embodiments of the present disclosure, and are not intended to limit the disclosure.

### EXAMPLE 1

### Materials and Methods

Formalin-fixed, paraffin-embedded (FFPE) tissue samples were obtained from the Pathology Department of Memorial Sloan-Kettering Cancer Center (MSKCC). The samples consist of unmounted 15 micron microtome slices taken from recently archived tumor tissue blocks. In most cases DNA was prepared between three and six months of original processing of the tissue for clinical histopathology. In all cases, samples were selected, prepared and coded at MSKCC before being sent to CSHL for ROMA analysis. Samples were given a score for amplification based on ROMA results and those scores were compared to the original pathology laboratory results at MSKCC. No additional patient identification or other clinical information was transferred to CSHL in this study.

ROMA and IHC/FISH results were compared between two different sample sets. The first set (Set A) was made up of 25 samples selected to contain a larger proportion of IHC+/FISH+ cases than would be present in a random set of patients. Set A was also selected to represent a range of ERBB2 amplification levels and tumor/normal tissue ratios in order to test the range of the ROMA technique. Set B was chronologically accumulated over a four-month period without regard to Her-2 status. The scores for ERBB2 amplification as measured by FISH and by ROMA were compared after 50, 75 and 100 samples were processed by ROMA.

The accumulated data for Set A and Set B are presented in Table 1. The observed amplification as detected by FISH was obtained by standard methods using the VySis system. ROMA was performed using a 390K array by methods described previously (e.g., Lucito et al. 2003). The raw values for scoring the level of amplification at ERBB2 (and other loci) were obtained by averaging the levels for the two highest scoring probes among the six probes on the array that detect the ROMA fragments overlapping both the long and short form of the ERBB2 gene. The raw values were translated into estimates of the amplification level. The translation of raw values into copy number was based on the experience in previous ROMA studies with amplicons that have been validated by FISH.

TABLE 1

| | | ROMA | | FISH | ROMA | ROMA | |
|---|---|---|---|---|---|---|---|
| MSKCC | | EXPT | TUM % | OBS.AMP | RAW.VAL | EST.AMP | NOTES |
| SET A | | | | | | | |
| BTL | 1 | MR669 | 70 | 5.2 | 1.35 | 3-4 | |
| BTL | 2 | MR671 | 90 | 8.5 | 2.31 | 6-8 | |
| BTL | 3 | MR641 | 90 | NORM | 1.03 | NORM | poss 17 p del |
| BTL | 4 | MR647 | 90 | 5.8 | 2.25 | 4-6 | narrow ERB |
| | | | | | | | peak |
| BTL | 5 | MR657 | 90 | 8.4 | 3.68 | 8-10 | |
| BTL | 6 | MR659 | 85 | 6.3 | 2.92 | 6-8 | |
| BTL | 7 | MR665 | 85 | 5.4 | 4.29 | 8-10 | |
| BTL | 8 | MR667 | 80 | 4.2 | 1.52 | 3-4 | |
| BTL | 9 | MR709 | 90 | NORM | 1.01 | NORM | |
| BTL | 10 | MR693 | 80 | 2.5 | 1.6 | 2-3 | |
| BTL | 11 | MR681 | 95 | 3 | 1.87 | 3-4 | |
| BTL | 12 | MR683 | 90 | 5.3 | 2.68 | 6-8 | |
| BTL | 13 | MR685 | >95 | 2.5 | 1.57 | 2-3 | |
| BTL | 14 | MR711 | 95 | NORM | 1.02 | NORM | |
| BTL | 15 | MR687 | 85 | 5.1 | 2.11 | 4-6 | |
| BTL | 16 | MR717 | 20 | 3.9 | .99 | NORM | POOR |
| | | | | | | | SIGNAL |
| BTL | 17 | MR719 | 80 | 7.1 | 2.0 | 3-6 | |
| BTL | 18 | MR689 | 80 | 2.2 | 1.37 | 1.5-2 | ARM |
| | | | | | | | DUPE |
| BTL | 19 | MR661 | 95 | 14.5 | 2.42 | 4-6 | |
| BTL | 20 | MR663 | 95 | 5.3 | 5.5 | 10+ | |
| BTL | 21 | MR691 | 95 | NORM | 1.0 | NORM | |
| BTL | 22 | MR721 | 75 | 6.2 | 2.85 | 6-8 | |
| BTL | 23 | MR723 | 80 | 5.1 | 2.32 | 4-6 | |
| BTL | 24 | MR725 | >95 | 9.2 | 3.21 | 8-10 | |
| BTL | 25 | MR727 | >95 | 5.7 | 2.49 | 4-6 | |
| | | | | FISH | ROMA | ROMA | |
| MSKCC | | EXPT | TUM % | ERB2 | RAW.VAL | EST.AMP | NOTES |
| SET B | | | | | | | |
| BTN | 5 | MR925 | | 10.4 | 10 | 8-10 | |
| BTN | 35 | MR937 | | 4.6 | 1.61 | 2-3 | |
| BTN | 39 | MR1053 | | 1.8 | 1.08 | NORM | |
| BTN | 45 | MR965 | | 5.5 | 4 | 4-5 | |
| BTN | 48 | MR971 | | 2.3 | 1 | NORM | FS on 17, |
| | | | | | | | not ERBB2 |
| BTN | 49 | MR973 | | 6 | 2.5 | 4-6 | |
| BTN | 63 | MR999 | | 1.8 | 1.6 | 2-3 | |
| BTN | 67 | MR1007 | | 11.8 | 10.1 | 10+ | |
| BTN | 73 | MR1181 | | 9.6 | 3 | 4-6 | |
| BTN | 76 | MR1187 | | 3.3 | 2.5 | 3-4 | |
| BTN | 79 | MR1193 | | 5.5 | 2.8 | 3-4 | |
| BTN | 81 | MR1197 | | 6.1 | 2.3 | 4-6 | ERBB2 |
| | | | | | | | broken |
| BTN | 82 | MR1199 | | 5 | 3.5 | 4-6 | |
| BTN | 84 | MR1227 | | 4.8 | 4 | 4-6 | |
| BTN | 98 | MR1257 | | 7.8 | 4 | 4-6 | |
| BTN | 101 | MR1275 | | 2.8 | 3.5 | 4-6 | |
| BTN | 102 | MR1263 | | 7 | 2.5 | 3-4 | ERBB2 |
| | | | | | | | broken |

### EXAMPLE 2

### Probe Design for FISH

Hybridization probes for FISH were constructed in one of two methods. For the interdigitation analysis, probes were created from bacterial artificial chromosomes (BAC) selected using the UCSD Genome Browser. For the determination of copy number in the deletions and amplifications of the aneuploid tumors, probes were made with PCR amplification of primers identified through the PROBER algorithm designed in this laboratory (Navin et al. 2006). Genomic sequences of 100 kb containing target amplifications were tiled with 50 probes (800-1400 bp).

Oligonucleotide primers were ordered in 96-well plates from Sigma Genosys and resuspended to 25 μM. Probes were amplified with the PCR Mastermix kit from Eppendorf (Cat. 0,032,002.447) from EBV immortalized cell line DNA (Chp-Skn-1) DNA (100 ng) with 55° C. annealing, 72° C. extension, 2 min extension time, and 23 cycles. Probes were purified with Qiagen PCR purification columns (Cat. 28,104) and combined into a single probe cocktail (10-25 μg total probes) for dye labeling and Metaphase/Interphase FISH.

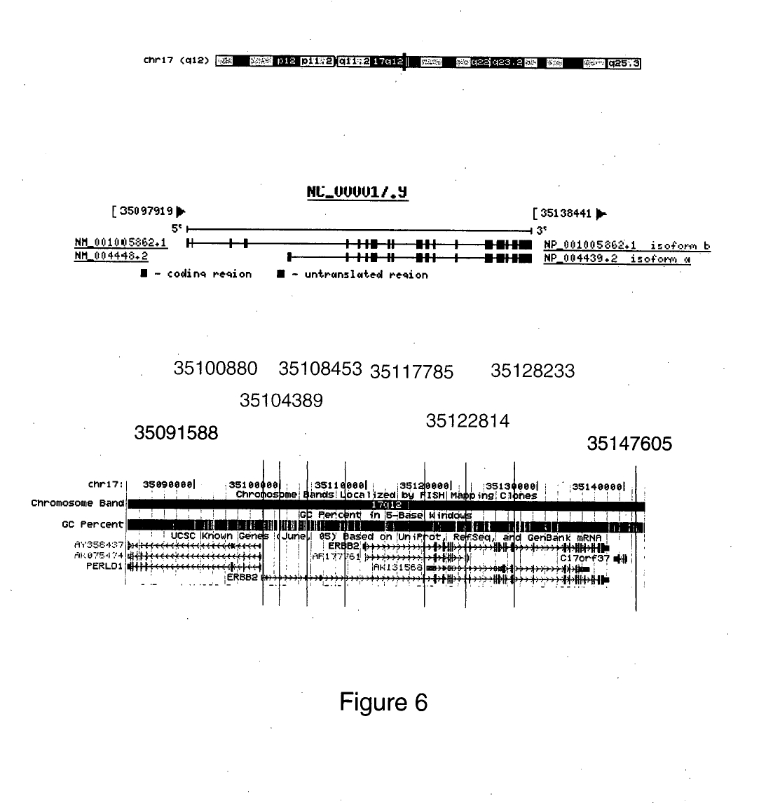

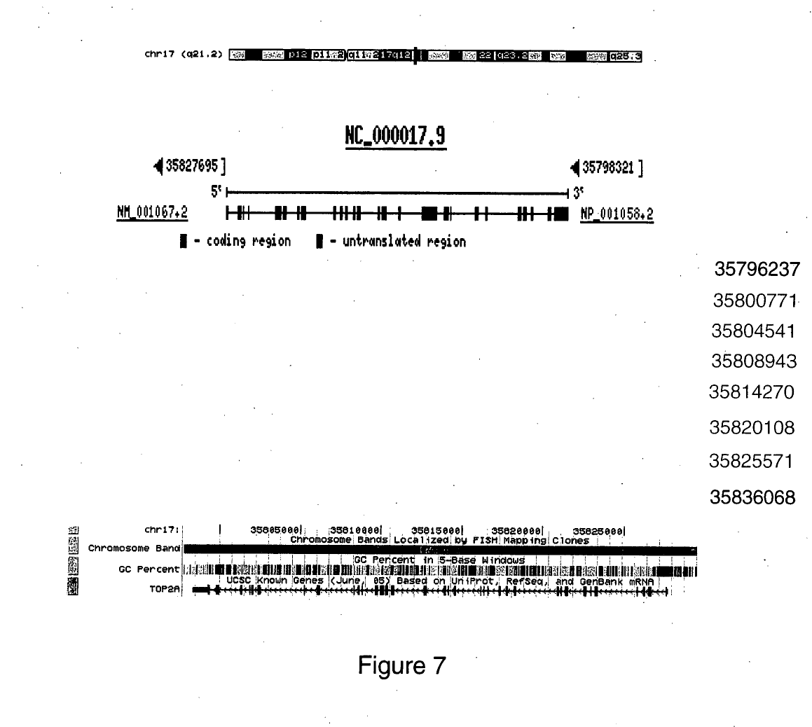

Certain probes (for the HER-2 locus and its neighboring or linked region) illustrated herein include 65-mer sequences homologous to the chromosomal regions between 35.050 Mb and 35.065 Mb, and between 35.09 Mb and 35.138 Mb of the human chromosome 17. The chromosomal positions of certain probes are also illustrated, e.g., in FIG. 6, in which the vertical lines indicate the probe positions.

### EXAMPLE 3

### ROMA Probes for Chromosome 17

Detailed information about ROMA probes used in the present and related studies has been made available to the public, such as for example at the ROMA web site (roma.cshl.edu). The follow table provides a first list of exemplary ROMA probes for detecting deletion events specific to chromosome 17 and a second list of exemplary ROMA probes for detecting amplification events specific to chromosome 17. The probes are part of the 85K ROMA array. Probes for a 390K ROMA array have also been made. Probes specific to other human chromosomes have also been made available to the public, for example, at the ROMA web site. See, e.g., Healy et al. 2003. Annotating large genomes with exact word matches. Genome Res. 2003 October;13(10):2306-15. Epub 2003 Sep 15 (describing a method for designing probes to chromosomal regions to be used in ROMA); see also U.S. Patent Application Publication No. 2005/0032095 (disclosing a word counting algorithm that can quickly and accurately count the number of times a particular string of characters, such as nucleotides, appears in a genomic nucleotide sequence); each of which are incorporated herein by reference. One of skill in the art using these and other known algorithms and methods need only specify the particular region for which the probe(s) need to be designed to generate set endpoints.

TABLE 2

| row.names | probe | CHROM | CHROM.POS | FRAG.POS | ABS.POS | density |
|---|---|---|---|---|---|---|
| X69427 | 69427 | 17 | 0.008308 | 7933 | 2486.625697 | 0.03195518 |
| X69507 | 69507 | 17 | 4.374241 | 4374213 | 2490.99163 | 0.03195518 |
| X69508 | 69508 | 17 | 4.508514 | 4508111 | 2491.125903 | 0.03077314 |
| X69521 | 69521 | 17 | 4.944597 | 4944384 | 2491.561986 | 0.03077314 |
| X69522 | 69522 | 17 | 4.946761 | 4946676 | 2491.56415 | 0.02957983 |
| X69527 | 69527 | 17 | 5.140134 | 5139817 | 2491.757523 | 0.02957983 |
| X69528 | 69528 | 17 | 5.16013 | 5160044 | 2491.777519 | 0.03181197 |
| X69567 | 69567 | 17 | 6.524363 | 6523336 | 2493.141752 | 0.03181197 |
| X69568 | 69568 | 17 | 6.584338 | 6583923 | 2493.201727 | 0.04161589 |
| X69669 | 69669 | 17 | 9.759236 | 9759047 | 2496.376625 | 0.04161589 |
| X69670 | 69670 | 17 | 9.767707 | 9767340 | 2496.385096 | 0.03181197 |
| X69674 | 69674 | 17 | 9.789433 | 9788802 | 2496.406822 | 0.03181197 |
| X69675 | 69675 | 17 | 9.834901 | 9833791 | 2496.45229 | 0.03109358 |
| X69694 | 69694 | 17 | 10.220993 | 10220337 | 2496.838382 | 0.03109358 |
| X69695 | 69695 | 17 | 10.292006 | 10291953 | 2496.909395 | 0.03687393 |
| X69744 | 69744 | 17 | 11.456257 | 11455703 | 2498.073646 | 0.03687393 |
| X69745 | 69745 | 17 | 11.490353 | 11489513 | 2498.107742 | 0.0596012 |
| X69777 | 69777 | 17 | 12.364404 | 12363962 | 2498.981793 | 0.0596012 |
| X69778 | 69778 | 17 | 12.37421 | 12373925 | 2498.991599 | 0.05791064 |
| X69788 | 69788 | 17 | 12.494377 | 12493786 | 2499.111766 | 0.05791064 |
| X69789 | 69789 | 17 | 12.519306 | 12519225 | 2499.136695 | 0.03518337 |
| X69812 | 69812 | 17 | 13.052002 | 13051646 | 2499.669391 | 0.03518337 |
| X69813 | 69813 | 17 | 13.060408 | 13060251 | 2499.677797 | 0.03460997 |
| X69859 | 69859 | 17 | 14.32544 | 14325144 | 2500.942829 | 0.03460997 |
| X69860 | 69860 | 17 | 14.364446 | 14364356 | 2500.981835 | 0.03173361 |
| X69867 | 69867 | 17 | 14.493189 | 14493070 | 2501.110578 | 0.03173361 |
| X69868 | 69868 | 17 | 14.535488 | 14535205 | 2501.152877 | 0.02595326 |
| X69881 | 69881 | 17 | 14.766493 | 14766442 | 2501.383882 | 0.02595326 |
| X69882 | 69882 | 17 | 14.864271 | 14863688 | 2501.48166 | 0.05720326 |
| X69909 | 69909 | 17 | 16.356679 | 16355927 | 2502.974068 | 0.05720326 |
| X69910 | 69910 | 17 | 16.383426 | 16383219 | 2503.000815 | 0.05634783 |
| X69913 | 69913 | 17 | 16.460839 | 16460575 | 2503.078228 | 0.05634783 |
| X69914 | 69914 | 17 | 16.805865 | 16805155 | 2503.423254 | 0.02509783 |
| X69915 | 69915 | 17 | 16.808693 | 16808449 | 2503.426082 | 0.02509783 |
| X69916 | 69916 | 17 | 16.845468 | 16845018 | 2503.462857 | 0.02305284 |
| X69950 | 69950 | 17 | 18.579279 | 18579109 | 2505.196668 | 0.02305284 |
| X69951 | 69951 | 17 | 18.695058 | 18694253 | 2505.312447 | 0.02221672 |
| X69955 | 69955 | 17 | 19.008758 | 19008558 | 2505.626147 | 0.02221672 |
| X69956 | 69956 | 17 | 19.066374 | 19066329 | 2505.683763 | 0.02032636 |
| X69957 | 69957 | 17 | 19.096491 | 19096276 | 2505.71388 | 0.02032636 |
| X69958 | 69958 | 17 | 19.293186 | 19293003 | 2505.910575 | 0.01979275 |
| X69971 | 69971 | 17 | 20.06781 | 20067110 | 2506.685199 | 0.01979275 |
| X69972 | 69972 | 17 | 20.092211 | 20091947 | 2506.7096 | 0.01926168 |
| X69974 | 69974 | 17 | 20.51325 | 20512949 | 2507.130639 | 0.01926168 |
| X69975 | 69975 | 17 | 20.559616 | 20559593 | 2507.177005 | 0.01743686 |
| X6997538 | 69975 | 17 | 20.559616 | 20559593 | 2507.177005 | 0.01743686 |
| X69976 | 69976 | 17 | 20.641486 | 20641311 | 2507.258875 | 0.01520472 |
| X69980 | 69980 | 17 | 20.857073 | 20856783 | 2507.474462 | 0.01520472 |
| X69981 | 69981 | 17 | 20.868269 | 20867928 | 2507.485658 | 0.01467618 |
| X69988 | 69988 | 17 | 21.208425 | 21208393 | 2507.825814 | 0.01467618 |
| X69989 | 69989 | 17 | 21.308585 | 21308379 | 2507.925974 | 0.02267618 |
| X6998939 | 69989 | 17 | 21.308585 | 21308379 | 2507.925974 | 0.02267618 |
| X69990 | 69990 | 17 | 21.319589 | 21318970 | 2507.936978 | 0.02370656 |
| X6999040 | 69990 | 17 | 21.319589 | 21318970 | 2507.936978 | 0.02370656 |
| X69991 | 69991 | 17 | 21.336943 | 21336685 | 2507.954332 | 0.02193351 |
| X69992 | 69992 | 17 | 21.615655 | 21614722 | 2508.233044 | 0.02193351 |
| X69993 | 69993 | 17 | 21.620471 | −1 | 2508.23786 | 0.01965382 |
| X69995 | 69995 | 17 | 22.025235 | 22025185 | 2508.642624 | 0.01965382 |
| X69996 | 69996 | 17 | 22.581276 | 22581112 | 2509.198665 | 0.01671953 |
| X70066 | 70066 | 17 | 24.912273 | 24912223 | 2511.529662 | 0.01671953 |
| X70067 | 70067 | 17 | 24.913451 | 24912591 | 2511.53084 | 0.02572853 |
| X70082 | 70082 | 17 | 25.553287 | 25553074 | 2512.170676 | 0.02572853 |
| X70083 | 70083 | 17 | 25.566639 | 25566277 | 2512.184028 | 0.02836706 |
| X70107 | 70107 | 17 | 26.72888 | 26728730 | 2513.346269 | 0.02836706 |
| X70108 | 70108 | 17 | 26.820939 | 26820591 | 2513.438328 | 0.02907178 |
| X70113 | 70113 | 17 | 26.858101 | 26857997 | 2513.47549 | 0.02907178 |
| X70114 | 70114 | 17 | 26.949618 | 26949210 | 2513.567007 | 0.02107178 |
| X70127 | 70127 | 17 | 27.88444 | 27884265 | 2514.501829 | 0.02107178 |
| X70128 | 70128 | 17 | 28.0567 | 28056172 | 2514.674089 | 0.1119809 |
| X70129 | 70129 | 17 | 28.060272 | 28059900 | 2514.677661 | 0.1119809 |
| X70130 | 70130 | 17 | 28.149389 | 28149220 | 2514.766778 | 0.1173 |
| X70138 | 70138 | 17 | 28.369171 | 28369063 | 2514.98656 | 0.1173 |
| X70139 | 70139 | 17 | 28.457558 | 28457020 | 2515.074947 | 0.02639093 |
| X70177 | 70177 | 17 | 28.962923 | 28962797 | 2515.580312 | 0.02639093 |
| X70178 | 70178 | 17 | 29.059737 | 29059693 | 2515.677126 | 0.07988191 |
| X70193 | 70193 | 17 | 29.333378 | 29333213 | 2515.950767 | 0.07988191 |
| X70194 | 70194 | 17 | 29.471091 | 29470461 | 2516.08848 | 0.01738192 |
| X70215 | 70215 | 17 | 29.979468 | 29979361 | 2516.596857 | 0.01738192 |
| X70216 | 70216 | 17 | 29.99608 | 29995969 | 2516.613469 | 0.01852608 |
| X70298 | 70298 | 17 | 32.530754 | 32530484 | 2519.148143 | 0.01852608 |
| X70299 | 70299 | 17 | 32.705491 | 32705343 | 2519.32288 | 0.01999021 |
| X70317 | 70317 | 17 | 33.254561 | 33254436 | 2519.87195 | 0.01999021 |
| X70318 | 70318 | 17 | 33.641819 | 33641436 | 2520.259208 | 0.01467106 |
| X70339 | 70339 | 17 | 34.595043 | 34594519 | 2521.212432 | 0.01467106 |
| X70340 | 70340 | 17 | 34.622355 | 34622177 | 2521.239744 | 0.01422721 |
| X70341 | 70341 | 17 | 34.637461 | 34637202 | 2521.25485 | 0.01422721 |
| X70342 | 70342 | 17 | 34.786206 | 34785966 | 2521.403595 | 0.01313432 |
| X70360 | 70360 | 17 | 35.245713 | 35245698 | 2521.863102 | 0.01313432 |
| X70361 | 70361 | 17 | 35.308957 | 35308426 | 2521.926346 | 0.0177747 |
| X70367 | 70367 | 17 | 35.597981 | 35597617 | 2522.21537 | 0.0177747 |
| X70368 | 70368 | 17 | 35.717975 | 35717638 | 2522.335364 | 0.02355505 |
| X70391 | 70391 | 17 | 36.13416 | 36134138 | 2522.751549 | 0.02355505 |
| X70392 | 70392 | 17 | 36.143756 | 36143018 | 2522.761145 | 0.0275233 |
| X70461 | 70461 | 17 | 38.424182 | 38423522 | 2525.041571 | 0.0275233 |
| X70462 | 70462 | 17 | 38.480675 | 38479881 | 2525.098064 | 0.02488478 |
| X70540 | 70540 | 17 | 41.29788 | 41297852 | 2527.915269 | 0.02488478 |
| X70541 | 70541 | 17 | 41.468279 | 41468013 | 2528.085668 | 0.01910443 |
| X70561 | 70561 | 17 | 42.439065 | 42438348 | 2529.056454 | 0.01910443 |
| X70562 | 70562 | 17 | 42.64482 | 42644287 | 2529.262209 | 0.01735618 |
| X70620 | 70620 | 17 | 43.969309 | 43969186 | 2530.586698 | 0.01735618 |
| X70621 | 70621 | 17 | 44.019754 | 44019683 | 2530.637143 | 0.01661544 |
| X70643 | 70643 | 17 | 44.588791 | 44588152 | 2531.20618 | 0.01661544 |
| X70644 | 70644 | 17 | 44.669729 | 44669256 | 2531.287118 | 0.01264718 |
| X70670 | 70670 | 17 | 45.499914 | 45499843 | 2532.117303 | 0.01264718 |
| X70671 | 70671 | 17 | 45.525305 | 45525172 | 2532.142694 | 0.01400403 |
| X70687 | 70687 | 17 | 45.978045 | 45978044 | 2532.595434 | 0.01400403 |
| X70688 | 70688 | 17 | 46.007036 | 46006672 | 2532.624425 | 0.01094593 |
| X7068841 | 70688 | 17 | 46.007036 | 46006672 | 2532.624425 | 0.01094593 |
| X70689 | 70689 | 17 | 46.040735 | 46039928 | 2532.658124 | 0.009601357 |
| X70753 | 70753 | 17 | 48.127958 | 48127712 | 2534.745347 | 0.009601357 |
| X70754 | 70754 | 17 | 48.156371 | 48156073 | 2534.77376 | 0.01378546 |
| X70888 | 70888 | 17 | 52.241489 | 52241189 | 2538.858878 | 0.01378546 |
| X70889 | 70889 | 17 | 52.25121 | 52251148 | 2538.868599 | 0.03339329 |
| X70939 | 70939 | 17 | 53.363965 | 53363715 | 2539.981354 | 0.03339329 |
| X70940 | 70940 | 17 | 53.473614 | 53473013 | 2540.091003 | 0.01378546 |
| X70981 | 70981 | 17 | 54.962169 | 54961998 | 2541.579558 | 0.01378546 |
| X70982 | 70982 | 17 | 54.990177 | 54989971 | 2541.607566 | 0.01232133 |
| X70992 | 70992 | 17 | 55.834018 | 55833898 | 2542.451407 | 0.01232133 |
| X70993 | 70993 | 17 | 55.9477 | 55947367 | 2542.565089 | 0.00655495 |
| X71006 | 71006 | 17 | 56.583137 | 56583052 | 2543.200526 | 0.00655495 |
| X71007 | 71007 | 17 | 56.600423 | 56600402 | 2543.217812 | 0.06537847 |
| X71023 | 71023 | 17 | 57.012081 | 57011860 | 2543.62947 | 0.06537847 |
| X71024 | 71024 | 17 | 57.029714 | 57029250 | 2543.647103 | 0.00655495 |
| X71074 | 71074 | 17 | 59.755544 | 59755165 | 2546.372933 | 0.00655495 |
| X71075 | 71075 | 17 | 59.927366 | 59926981 | 2546.544755 | 0.006196783 |
| X71086 | 71086 | 17 | 60.366443 | 60366332 | 2546.983832 | 0.006196783 |
| X71087 | 71087 | 17 | 60.445112 | 60444935 | 2547.062501 | 0.01260704 |
| X71089 | 71089 | 17 | 60.524308 | 60524004 | 2547.141697 | 0.01260704 |
| X71090 | 71090 | 17 | 60.569096 | 60568555 | 2547.186485 | 0.01146287 |
| X71229 | 71229 | 17 | 65.586483 | 65586440 | 2552.203872 | 0.01146287 |
| X71230 | 71230 | 17 | 65.588212 | 65588012 | 2552.205601 | 0.01646288 |
| X71242 | 71242 | 17 | 66.012044 | 66011956 | 2552.629433 | 0.01646288 |
| X71243 | 71243 | 17 | 66.069234 | 66068477 | 2552.686623 | 0.01005262 |
| X71274 | 71274 | 17 | 66.994082 | 66993967 | 2553.611471 | 0.01005262 |
| X71275 | 71275 | 17 | 67.017015 | 67016856 | 2553.634404 | 0.01757142 |
| X71310 | 71310 | 17 | 67.668955 | 67668783 | 2554.286344 | 0.01757142 |
| X71311 | 71311 | 17 | 67.69516 | 67694744 | 2554.312549 | 0.02220104 |
| X71365 | 71365 | 17 | 69.160866 | 69160612 | 2555.778255 | 0.02220104 |
| X71366 | 71366 | 17 | 69.189366 | 69189137 | 2555.806755 | 0.03230205 |
| X71407 | 71407 | 17 | 70.817127 | 70816273 | 2557.434516 | 0.03230205 |
| X71408 | 71408 | 17 | 70.829309 | 70829095 | 2557.446698 | 0.02342641 |
| X71429 | 71429 | 17 | 72.004052 | 72003984 | 2558.621441 | 0.02342641 |
| X71430 | 71430 | 17 | 72.237388 | 72236538 | 2558.854777 | 0.01842641 |
| X71464 | 71464 | 17 | 73.765605 | 73765224 | 2560.382994 | 0.01842641 |
| X71465 | 71465 | 17 | 73.812268 | 73812230 | 2560.429657 | 0.008325396 |
| X71466 | 71466 | 17 | 73.81353 | 73813314 | 2560.430919 | 0.008325396 |
| X71467 | 71467 | 17 | 73.921462 | −1 | 2560.538851 | 0.02499206 |
| X71512 | 71512 | 17 | 76.814261 | 76814104 | 2563.43165 | 0.02499206 |
| X71513 | 71513 | 17 | 76.951018 | 76950595 | 2563.568407 | 0.09642063 |
| X71526 | 71526 | 17 | 78.56987 | 78569587 | 2565.187259 | 0.09642063 |
| X69427 | 69427 | 17 | 0.008308 | 7933 | 2486.625697 | 0.001146789 |
| X69519 | 69519 | 17 | 4.858229 | 4857865 | 2491.475618 | 0.001146789 |
| X69520 | 69520 | 17 | 4.932018 | 4931969 | 2491.549407 | 0.02198012 |
| X69567 | 69567 | 17 | 6.524363 | 6523336 | 2493.141752 | 0.02198012 |
| X69568 | 69568 | 17 | 6.584338 | 6583923 | 2493.201727 | 0.001146789 |
| X69859 | 69859 | 17 | 14.32544 | 14325144 | 2500.942829 | 0.001146789 |
| X69860 | 69860 | 17 | 14.364446 | 14364356 | 2500.981835 | 0.09205588 |
| X69881 | 69881 | 17 | 14.766493 | 14766442 | 2501.383882 | 0.09205588 |
| X69882 | 69882 | 17 | 14.864271 | 14863688 | 2501.48166 | 0.001146789 |
| X69955 | 69955 | 17 | 19.008758 | 19008558 | 2505.626147 | 0.001146789 |
| X69956 | 69956 | 17 | 19.066374 | 19066329 | 2505.683763 | 0.03055855 |
| X69957 | 69957 | 17 | 19.096491 | 19096276 | 2505.71388 | 0.03055855 |
| X69958 | 69958 | 17 | 19.293186 | 19293003 | 2505.910575 | 0.03082773 |
| X69971 | 69971 | 17 | 20.06781 | 20067110 | 2506.685199 | 0.03082773 |
| X69972 | 69972 | 17 | 20.092211 | 20091947 | 2506.7096 | 0.03147082 |
| X69974 | 69974 | 17 | 20.51325 | 20512949 | 2507.130639 | 0.03147082 |
| X69975 | 69975 | 17 | 20.559616 | 20559593 | 2507.177005 | 0.1028994 |
| X69988 | 69988 | 17 | 21.208425 | 21208393 | 2507.825814 | 0.1028994 |
| X69989 | 69989 | 17 | 21.308585 | 21308379 | 2507.925974 | 0.03147082 |
| X6998938 | 69989 | 17 | 21.308585 | 21308379 | 2507.925974 | 0.03147082 |
| X69990 | 69990 | 17 | 21.319589 | 21318970 | 2507.936978 | 0.002059055 |
| X69992 | 69992 | 17 | 21.615655 | 21614722 | 2508.233044 | 0.002059055 |
| X69993 | 69993 | 17 | 21.620471 | −1 | 2508.23786 | 0.01075471 |
| X69994 | 69994 | 17 | 21.895896 | 21895317 | 2508.513285 | 0.01075471 |
| X69995 | 69995 | 17 | 22.025235 | 22025185 | 2508.642624 | 0.01665797 |
| X6999539 | 69995 | 17 | 22.025235 | 22025185 | 2508.642624 | 0.01665797 |
| X69996 | 69996 | 17 | 22.581276 | 22581112 | 2509.198665 | 0.01981113 |
| X6999640 | 69996 | 17 | 22.581276 | 22581112 | 2509.198665 | 0.01981113 |
| X69997 | 69997 | 17 | 22.641471 | 22641339 | 2509.25886 | 0.02732993 |
| X70107 | 70107 | 17 | 26.72888 | 26728730 | 2513.346269 | 0.02732993 |
| X70108 | 70108 | 17 | 26.820939 | 26820591 | 2513.438328 | 0.01863428 |
| X70113 | 70113 | 17 | 26.858101 | 26857997 | 2513.47549 | 0.01863428 |
| X70114 | 70114 | 17 | 26.949618 | 2694210 | 2513.567007 | 0.09006285 |
| X70127 | 70127 | 17 | 27.88444 | 27884265 | 2514.501829 | 0.09006285 |
| X70128 | 70128 | 17 | 28.0567 | 28056172 | 2514.674089 | 0.01863428 |
| X70129 | 70129 | 17 | 28.060272 | 28059900 | 2514.677661 | 0.01863428 |
| X70130 | 70130 | 17 | 28.149389 | 28149220 | 2514.766778 | 0.01111548 |
| X70138 | 70138 | 17 | 28.369171 | 28369063 | 2514.98656 | 0.01111548 |
| X70139 | 70139 | 17 | 28.457558 | 28457020 | 2515.074947 | 0.0367565 |
| X70177 | 70177 | 17 | 28.962923 | 28962797 | 2515.580312 | 0.0367565 |
| X70178 | 70178 | 17 | 29.059737 | 29059693 | 2515.677126 | 0.03743127 |
| X70193 | 70193 | 17 | 29.333378 | 29333213 | 2515.950767 | 0.03743127 |
| X70194 | 70194 | 17 | 29.471091 | 29470461 | 2516.08848 | 0.04317839 |
| X70203 | 70203 | 17 | 29.641228 | 29640958 | 2516.258617 | 0.04317839 |
| X70204 | 70204 | 17 | 29.69944 | 29699403 | 2516.316829 | 0.03839371 |
| X70215 | 70215 | 17 | 29.979468 | 29979361 | 2516.596857 | 0.03839371 |
| X70216 | 70216 | 17 | 29.99608 | 29995969 | 2516.613469 | 0.01207792 |
| X70289 | 70289 | 17 | 32.243258 | 32243258 | 2518.860647 | 0.01207792 |
| X70290 | 70290 | 17 | 32.279846 | 32279626 | 2518.897235 | 0.01693229 |
| X70298 | 70298 | 17 | 32.530754 | 32530484 | 2519.148143 | 0.01693229 |
| X70299 | 70299 | 17 | 32.705491 | 32705343 | 2519.32288 | 0.0157855 |
| X70317 | 70317 | 17 | 33.254561 | 33254436 | 2519.87195 | 0.0157855 |
| X70318 | 70318 | 17 | 33.641819 | 33641436 | 2520.259208 | 0.03904131 |
| X7031841 | 70318 | 17 | 33.641819 | 33641436 | 2520.259208 | 0.03904131 |
| X70319 | 70319 | 17 | 33.679199 | 33679164 | 2520.296588 | 0.03986913 |
| X70323 | 70323 | 17 | 33.708088 | 33707897 | 2520.325477 | 0.03986913 |
| X70324 | 70324 | 17 | 34.082394 | 34081943 | 2520.699783 | 0.05356776 |
| X70325 | 70325 | 17 | 34.156366 | 34155874 | 2520.773755 | 0.05356776 |
| X70326 | 70326 | 17 | 34.156623 | 34156444 | 2520.774012 | 0.06785347 |
| X70339 | 70339 | 17 | 34.595043 | 34594519 | 2521.212432 | 0.06785347 |
| X70340 | 70340 | 17 | 34.622355 | 34622177 | 2521.239744 | 0.113308 |
| X70341 | 70341 | 17 | 34.637461 | 34637202 | 2521.25485 | 0.113308 |
| X70342 | 70342 | 17 | 34.786206 | 34785966 | 2521.403595 | 0.183308 |
| X70360 | 70360 | 17 | 35.245713 | 35245698 | 2521.863102 | 0.183308 |
| X70361 | 70361 | 17 | 35.308957 | 35308426 | 2521.926346 | 0.1600522 |
| X7036142 | 70361 | 17 | 35.308957 | 35308426 | 2521.926346 | 0.1600522 |
| X70362 | 70362 | 17 | 35.383839 | 35383116 | 2522.001228 | 0.06459766 |
| X70367 | 70367 | 17 | 35.597981 | 35597617 | 2522.21537 | 0.06459766 |
| X70368 | 70368 | 17 | 35.717975 | 35717638 | 2522.335364 | 0.05885053 |
| X70391 | 70391 | 17 | 36.13416 | 36134138 | 2522.751549 | 0.05885053 |
| X70392 | 70392 | 17 | 36.143756 | 36143018 | 2522.761145 | 0.03885053 |
| X70395 | 70395 | 17 | 36.171421 | 36170809 | 2522.78881 | 0.03885053 |
| X70396 | 70396 | 17 | 36.2707 | 36270547 | 2522.888089 | 0.02206482 |
| X7039643 | 70396 | 17 | 36.2707 | 36270547 | 2522.888089 | 0.02206482 |
| X70397 | 70397 | 17 | 36.271448 | 36271429 | 2522.888837 | 0.008366185 |
| X70461 | 70461 | 17 | 38.424182 | 38423522 | 2525.041571 | 0.008366185 |
| X70462 | 70462 | 17 | 38.480675 | 38479881 | 2525.098064 | 0.009305152 |
| X70495 | 70495 | 17 | 39.617066 | 39616900 | 2526.234455 | 0.009305152 |
| X70496 | 70496 | 17 | 39.655922 | 39655701 | 2526.273311 | 0.004450784 |
| X70561 | 70561 | 17 | 42.439065 | 42438348 | 2529.056454 | 0.004450784 |
| X70562 | 70562 | 17 | 42.64482 | 42644287 | 2529.262209 | 0.005487053 |
| X70594 | 70594 | 17 | 43.321331 | 43321100 | 2529.93872 | 0.005487053 |
| X70595 | 70595 | 17 | 43.390642 | 43390416 | 2530.008031 | 0.0083268 |
| X70599 | 70599 | 17 | 43.470718 | 43470565 | 2530.088107 | 0.0083268 |
| X70600 | 70600 | 17 | 43.472024 | 43471659 | 2530.089413 | 0.01118808 |
| X70608 | 70608 | 17 | 43.671549 | 43671295 | 2530.288938 | 0.01118808 |
| X70609 | 70609 | 17 | 43.673855 | 43673783 | 2530.291244 | 0.01927041 |
| X70620 | 70620 | 17 | 43.969309 | 43969186 | 2530.586698 | 0.01927041 |
| X70621 | 70621 | 17 | 44.019754 | 44019683 | 2530.637143 | 0.03927041 |
| X70629 | 70629 | 17 | 44.292741 | 44292498 | 2530.91013 | 0.03927041 |
| X70630 | 70630 | 17 | 44.294746 | 44294620 | 2530.912135 | 0.05514342 |
| X70643 | 70643 | 17 | 44.588791 | 44588152 | 2531.20618 | 0.05514342 |
| X70644 | 70644 | 17 | 44.669729 | 44669256 | 2531.287118 | 0.07736181 |
| X70667 | 70667 | 17 | 45.336263 | 45336159 | 2531.953652 | 0.07736181 |
| X70668 | 70668 | 17 | 45.361237 | 45360777 | 2531.978626 | 0.08262496 |
| X70670 | 70670 | 17 | 45.499914 | 45499843 | 2532.117303 | 0.08262496 |
| X70671 | 70671 | 17 | 45.525305 | 45525172 | 2532.142694 | 0.06262495 |
| X70682 | 70682 | 17 | 45.871718 | 45870654 | 2532.489107 | 0.06262495 |
| X70683 | 70683 | 17 | 45.884537 | 45884421 | 2532.501926 | 0.065942 |
| X70688 | 70688 | 17 | 46.007036 | 46006672 | 2532.624425 | 0.065942 |
| X70689 | 70689 | 17 | 46.040735 | 46039928 | 2532.658124 | 0.06722734 |
| X7068944 | 70689 | 17 | 46.040735 | 46039928 | 2532.658124 | 0.06722734 |
| X70690 | 70690 | 17 | 46.181432 | 46181317 | 2532.798821 | 0.06842209 |
| X70691 | 70691 | 17 | 46.181588 | 46181563 | 2532.798977 | 0.06842209 |
| X70692 | 70692 | 17 | 46.235028 | 46234898 | 2532.852417 | 0.04758874 |
| X7069245 | 70692 | 17 | 46.235028 | 46234898 | 2532.852417 | 0.04758874 |
| X70693 | 70693 | 17 | 46.235938 | 46235567 | 2532.853327 | 0.03171573 |
| X70751 | 70751 | 17 | 48.00957 | 48009360 | 2534.626959 | 0.03171573 |
| X70752 | 70752 | 17 | 48.111083 | 48110923 | 2534.728472 | 0.02472273 |
| X70802 | 70802 | 17 | 50.319266 | 50319058 | 2536.936655 | 0.02472273 |
| X70803 | 70803 | 17 | 50.321274 | 50321273 | 2536.938663 | 0.02649892 |
| X70857 | 70857 | 17 | 51.450922 | 51449900 | 2538.068311 | 0.02649892 |
| X70858 | 70858 | 17 | 51.460364 | 51460071 | 2538.077753 | 0.02123577 |
| X70888 | 70888 | 17 | 52.241489 | 52241189 | 2538.858878 | 0.02123577 |
| X70889 | 70889 | 17 | 52.25121 | 52251148 | 2538.868599 | 0.0201172 |
| X70981 | 70981 | 17 | 54.962169 | 54961998 | 2541.579558 | 0.0201172 |
| X70982 | 70982 | 17 | 54.990177 | 54989971 | 2541.607566 | 0.02089967 |
| X70992 | 70992 | 17 | 55.834018 | 55833898 | 2542.451407 | 0.02089967 |
| X70993 | 70993 | 17 | 55.9477 | 55947367 | 2542.565089 | 0.09232824 |
| X71006 | 71006 | 17 | 56.583137 | 56583052 | 2543.200526 | 0.09232824 |
| X71007 | 71007 | 17 | 56.600423 | 56600402 | 2543.217812 | 0.02089967 |
| X71023 | 71023 | 17 | 57.012081 | 57011860 | 2543.62947 | 0.02089967 |
| X71024 | 71024 | 17 | 57.029714 | 57029250 | 2543.647103 | 0.03677268 |
| X71030 | 71030 | 17 | 57.417119 | 57417001 | 2544.034508 | 0.03677268 |
| X71031 | 71031 | 17 | 57.496449 | 57496141 | 2544.113838 | 0.04446499 |
| X71038 | 71038 | 17 | 57.718589 | 57718168 | 2544.335978 | 0.04446499 |
| X71039 | 71039 | 17 | 57.815751 | 57815727 | 2544.43314 | 0.05266171 |
| X71086 | 71086 | 17 | 60.366443 | 60366332 | 2546.983832 | 0.05266171 |
| X71087 | 71087 | 17 | 60.445112 | 60444935 | 2547.062501 | 0.03678869 |
| X71089 | 71089 | 17 | 60.524308 | 60524004 | 2547.141697 | 0.03678869 |
| X71090 | 71090 | 17 | 60.569096 | 60568555 | 2547.186485 | 0.03907702 |
| X71151 | 71151 | 17 | 63.052153 | 63051982 | 2549.669542 | 0.03907702 |
| X71152 | 71152 | 17 | 63.07899 | 63078517 | 2549.696379 | 0.03694483 |
| X71160 | 71160 | 17 | 63.660162 | 63659581 | 2550.277551 | 0.03694483 |
| X71161 | 71161 | 17 | 63.691599 | 63691278 | 2550.308988 | 0.01750649 |
| X71242 | 71242 | 17 | 66.012044 | 66011956 | 2552.629433 | 0.01750649 |
| X71243 | 71243 | 17 | 66.069234 | 66068477 | 2552.686623 | 0.02253161 |
| X71261 | 71261 | 17 | 66.609735 | 66608748 | 2553.227124 | 0.02253161 |
| X71262 | 71262 | 17 | 66.719652 | 66719641 | 2553.337041 | 0.0263052 |
| X71322 | 71322 | 17 | 67.89488 | 67893994 | 2554.512269 | 0.0263052 |
| X71323 | 71323 | 17 | 67.915736 | 67915706 | 2554.533125 | 0.03694349 |
| X71354 | 71354 | 17 | 68.703637 | 68703263 | 2555.321026 | 0.03694349 |
| X71355 | 71355 | 17 | 68.73665 | 68736385 | 2555.354039 | 0.04843774 |
| X71365 | 71365 | 17 | 69.160866 | 69160612 | 2555.778255 | 0.04843774 |
| X71366 | 71366 | 17 | 69.189366 | 69189137 | 2555.806755 | 0.0452765 |
| X71416 | 71416 | 17 | 71.556581 | 71555790 | 2558.17397 | 0.0452765 |
| X71417 | 71417 | 17 | 71.655102 | 71654939 | 2558.272491 | 0.03463821 |
| X71441 | 71441 | 17 | 72.781822 | 72781354 | 2559.399211 | 0.03463821 |
| X71442 | 71442 | 17 | 72.858469 | 72858251 | 2559.475858 | 0.01811883 |
| X71448 | 71448 | 17 | 73.148351 | 73148281 | 2559.76574 | 0.01811883 |
| X71449 | 71449 | 17 | 73.205916 | 73205519 | 2559.823305 | 0.03093935 |
| X71464 | 71464 | 17 | 73.765605 | 73765224 | 2560.382994 | 0.03093935 |
| X71465 | 71465 | 17 | 73.812268 | 73812230 | 2560.429657 | 0.05177267 |
| X71466 | 71466 | 17 | 73.81353 | 73813314 | 2560.430919 | 0.05177267 |
| X71467 | 71467 | 17 | 73.921462 | −1 | 2560.538851 | 0.05048732 |
| X71512 | 71512 | 17 | 76.814261 | 76814104 | 2563.43165 | 0.05048732 |
| X71513 | 71513 | 17 | 76.951018 | 76950595 | 2563.568407 | 0.029654 |
| X71526 | 71526 | 17 | 78.56987 | 78569587 | 2565.187259 | 0.029654 |

Legend for the columns:
probe - ROMA probe number;
CHROM - chromosome number;
CHROM.POS - probe start base pair position within the chromosome;
FRAG.POS - fragment start base pair position within the chromosome;
ABS.POS - absolute probe start base pair position;
density - event density measure as explained in the text. Data from the May 2004, Human Genome Sequence 17.

### EXAMPLE 3

### Correlation of FISH and ROMA in Selected Cases (SET A)

The data presented in Table 1 show a remarkably good agreement between FISH and ROMA data. The data for the predominantly Her-2+ cases in Set A in Table 1 show that all cases that scored negative by FISH were scored negative by ROMA (4/4). For the cases scored positive by FISH, all but one were detected by ROMA (20/21). Furthermore, with one exception (BTL19) the estimates for degree of amplification by the two techniques show a strong correlation. The single case that was scored as a false negative by ROMA (BTL16) had only 20% tumor cells and thus sets an apparent lower limit on the ratio of tumor/normal nuclei in the sample tissue for efficient detection by a mass technique such as ROMA.

### EXAMPLE 4

### Correlation of FISH and ROMA in Unselected Cases (SET B)

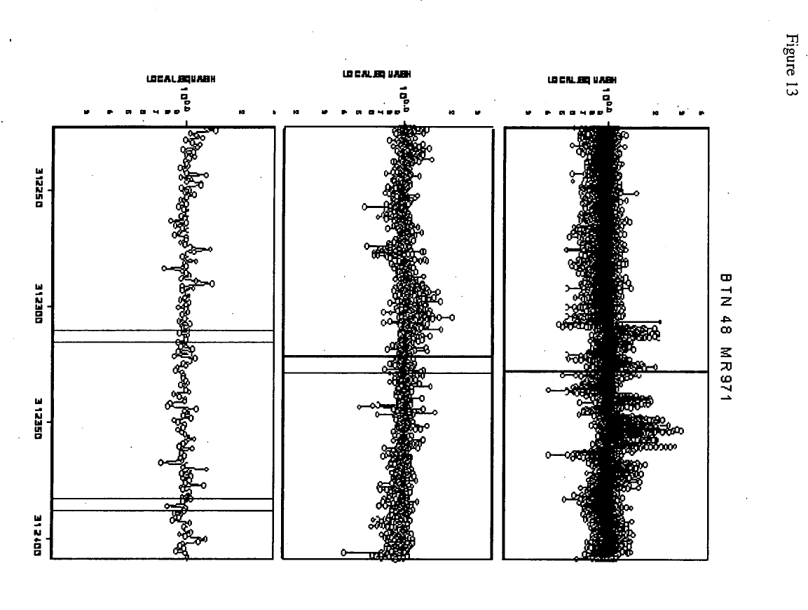

Table 1 also shows selected data derived from Set B, consisting of 102 cases accumulated sequentially and tested by ROMA. It is the practice at MSKCC that all tumors are assayed for Her-2 using immunohistochemistry (IHC) and only those scoring 2+ or 3+ at least in part of the tumor are further tested using FISH. Therefore, both IHC and ROMA were performed on all samples, while FISH was performed on a subset of the cases. Among the IHC positive cases there was a clear difference in the likelihood of observing amplification by either FISH or ROMA. All IHC3+ patients showed detectable amplification of the ERBB2 locus while only IHC2+ cases showed amplification. The correlation of scoring for the degree of amplification in each positive sample is shown by the graph in FIG. 13.

Complete data for all of the samples including the IHC negative samples is presented on the ROMA Web site associated with Cold Spring Harbor Laboratories (romafiles). The breakdown of the sample scoring for IHC, FISH and ROMA is presented in Table 3.

TABLE 3
Breakdown of cases according to IHC status

| | Set A | Set B | Notes |
|---|---|---|---|
| Total | | 102 | |
| IHC pos | 25 | [ ] | |
| (2+/3+) | | | |
| IHC pos & | 21 | 17 | 1 False positive for |
| FISH pos | | | FISH |
| IHC pos & | 20 | 15 | 1 False negative for |
| ROMA+ | | | ROMA |
| IHC neg & | NA | [ ] | No False positives |
| ROMA neg | | | for ROMA |

The samples shown in Table 1 for Set B were those for which FISH scored positive at a level of at least 1.5× normal genome copy number. Only one case, BTN39, scored positive for FISH (1.8×) and did not show a signal by ROMA. This counts as a false negative for ROMA by the currently used clinical criteria, but it is clearly a borderline case and it has not clear whether 1.8 fold amplification constitutes sufficient amplification for beneficial treatment targeting HER-2.

All cases scoring 2× or better by FISH (16/16) showed amplification around the ERBB2 locus in the ROMA profile. However, one of these cases, BTN48, was scored as NORMAL for Her-2 amplification in Table 1 (Set B) because the amplification observed by ROMA is adjacent to the ERBB2 locus but does not include the ERBB2 gene itself. The ROMA profile for this case, shown in FIG. 13, exhibits at least five amplicons on the 17q arm in this tumor, but a magnified view shows that the ERBB2 is located between two of those amplicons. Although the exact endpoints of the sequences included in the VySis FISH probe are proprietary, it is reasonable to assume that they extend far outside the boundaries of the target gene and therefore will occasionally score positive for an adjacent amplicon. This represents a type of systematic false positive for FISH.

### EXAMPLE 5

### Duplications of Chromosome 17q

Another event that can be detected by genome scanning methods such as ROMA are broad duplications of entire or nearly entire chromosome arms. Often these large duplications do not include the centromere and thus have to be maintained as translocations to other chromosomes. BTN18 (Table 1) is an example of such a duplication. The copy number relative to the chromsome 17 centromere is 2×, but the question arises as to whether this constitutes an amplification for the purpose of directing therapy. ERBB2 is doubled in copy number relative to the rest of the genome (or the parts which are not also duplicated) but not relative to its surrounding loci as is the case when narrow amplicons are formed.

All references cited herein are incorporated by reference in their entirety, including the following references:
Lakshmi B, Hall I M, Egan C, Alexander J, Leotta A, Healy J, Zender L, Spector M S, Xue W, Lowe S W, Wigler M, Lucito, “Mouse genomic representational oligonucleotide microarray analysis: detection of copy number variations in normal and tumor specimens,” Proc Natl Acad Sci USA. 2006 Jul. 25;103(30):11234-9. Epub 2006 Jul. 14.
Zender L, Spector M S, Xue W, Flemming P, Cordon-Cardo C, Silke J, Fan S T, Luk J M, Wigler M, Hannon G J, Mu D, Lucito R, Powers S, Lowe S W, “Identification and validation of oncogenes in liver cancer using an integrative oncogenomic approach,” Cell. 2006 Jun. 30;125(7):1253-67.
Daruwala R S, Rudra A, Ostrer H, Lucito R, Wigler M, Mishra B, “A versatile statistical analysis algorithm to detect genome copy number variation,” Proc Natl Acad Sci USA. 2004 Nov. 16;101(46):16292-7. Epub 2004 Nov. 8.
Olshen A B, Venkatraman E S, Lucito R, Wigler M, “Circular binary segmentation for the analysis of array-based DNA copy number data,” Biostatistics. 2004 October;5(4):557-72.
Sebat J, Lakshmi B, Troge J, Alexander J, Young J, Lundin P, Maner S, Massa H, Walker M, Chi M, Navin N, Lucito R, Healy J, Hicks J, Ye K, Reiner A, Gilliam T C, Trask B, Patterson N, Zetterberg A, Wigler M, “Large-scale copy number polymorphism in the human genome,”
Science. 2004 Jul 23;305(5683):525-8.
Lucito R, Healy J, Alexander J, Reiner A, Esposito D, Chi M, Rodgers L, Brady A, Sebat J, Troge J, West J A, Rostan S, Nguyen K C, Powers S, Ye K Q, Olshen A, Venkatraman E, Norton L, Wigler M, “Representational oligonucleotide microarray analysis: a high-resolution method to detect genome copy number variation,” Genome Res. 2003 October;13(10):2291-305. Epub 2003 Sep. 15.
Lucito R, West J, Reiner A, Alexander J, Esposito D, Mishra B, Powers S, Norton L, Wigler M, “Detecting gene copy number fluctuations in tumor cells by microarray analysis of genomic representations,” Genome Res. 2000 November;10(11):1726-36.
Lucito R, Nakimura M, West J A, Han Y, Chin K, Jensen K, McCombie R, Gray J W, Wigler M, “Genetic analysis using genomic representations,” Proc Natl Acad Sci USA. 1998 Apr. 14;95(8):4487-92.
Navin et al. (2006) PROBER: oligonucleotide FISH probe design software. Bioinfornatics. 2006 Oct. 1;22(19):2437-8. Epub 2006 Jun. 1.
Hicks et al. (2005) High-resolution ROMA CGH and FISH analysis of aneuploid and diploid breast tumors. Cold Spring Harb Symp Quant Biol. 2005;70:51-63.
Hicks et al. (2006) Novel patterns of genome rearrangement and their association with survival in breast cancer. Genome Res. 16(12): 1465-79.
U.S. Patent Application Publication No. 20050266444, “Use of representations of DNA for genetic analysis”;
U.S. Patent Application Publication No. 20050196799, “Use of representations of DNA for genetic analysis”;
U.S. Patent Application Publication No. 20050032095, “Virtual representations of nucleotide sequences”;
U.S. Patent Application Publication No. 20040197774, “Representational approach to DNA analysis”;

U.S. Patent Application Publication No. 20040137473, “Use of representations of DNA for genetic analysis.”

TABLE 4
Selected genes involved in breast cancer diagnosis or
susceptibility

| gene | chrom | band | probe | chrom.pos | probe | .width | | | |
|---|---|---|---|---|---|---|---|---|---|
| ESR1 | 6 | q25.1 | 34559 | 152.16 | 34565 | 152.47 | 6 | | |
| PGR | 11 | q22-q23 | 54025 | 100.42 | 54029 | 100.54 | 4 | | |
| HER2/neu | 17 | q21 | 70354 | 35.08 | 70355 | 35.2 | 2 | bracket | amp |
| TOP2A | 17 | q21-q22 | 70372 | 35.8 | 70373 | 35.84 | 2 | on gene | amp |
| ATM | 11 | q22-q23 | 54241 | 107.66 | 54243 | 107.83 | 3 | | del |
| CHEK1 | 11 | q24 | 54928 | 124.8 | 54929 | 125.04 | 2 | | del |
| EGFR1 | 7 | p12 | 36800 | 54.84 | 36806 | 55.06 | 7 | | |
| CCND | 11 | q | 53064 | 69.11 | 53065 | 69.18 | 2 | bracket | amp |
| MYC | 8 | q24.12 | 43267 | 128.75 | 43258 | 128.81 | 2 | bracket | amp |
| TP53 | 17 | p12 | 69589 | 7.5 | 69590 | 7.59 | 2 | bracket | del |
| NOG | | | | | | | | | |
| BRCA1 | 17 | q21 | 70461 | 38.42 | 70464 | 38.72 | 4 | bracket | |
| BRCA2 | 13 | q12.3 | 59605 | 31.77 | 59607 | 31.91 | 3 | on gene | |
| CDKN2A | 9 | p21 | 44459 | 21.96 | 44460 | 22.04 | 2 | bracket | del |
| CHEK1 | 22 | q11-12 | 78282 | 27.35 | 78283 | 27.47 | 2 | bracket | del |
| TOB1 | 17 | q21 | 70693 | 46.24 | 70694 | 46.44 | 2 | bracket | amp |
| CKS1 | 1 | q21.2 | 3850 | 151.75 | 3851 | 151.96 | 2 | bracket | amp |
| BCAS1 | 20 | q13.2-13.3 | 76564 | 51.99 | 76571 | 52.18 | 8 | on gene | |
| BCAR3 | 1 | p22.1 | 2788 | 93.64 | 2799 | 93.89 | 12 | on gene | del |
| BCAR1 | 16 | q22-23 | 68998 | 73.8 | 68999 | 73.89 | 2 | bracket | |
| HOXB grp | 17 | q21.3 | 70615 | 43.75 | 70624 | 44.11 | | bracket | |
| PI3K | 7 | q22.3 | 37878 | 106.1 | 37879 | 106.15 | 2 | on gene | |

We claim:

1. A method for assessing a patient's likely response to a genetic locus X-based therapy comprising:
(a) determining the copy number (x) of one or more segments of genomic DNA comprising a genetic locus X (X) relative to the copy number (r) of a linked chromosomal region (R) present in DNA extracted from one or more cancer cells of the patient;
(b) determining the copy number (i), relative to the copy number (r) of said region (R), of one or more segments of genomic DNA (I) interspersed between said genetic locus X and said region;
wherein the copy number (i) of said interspersed region (I) relative to that of either or both of said region (R) and said genetic locus X (X) determines the likely response of the patient to said genetic locus X-based therapy.

2. The method of claim 1, wherein said genetic locus X is a HER2 locus, and said therapy comprises treatment with Herceptin®.

3. The method of claim 1, wherein said genetic locus X is a TOP2A locus, and said therapy comprises a chemotherapy.

4. The method of claim 3, wherein said chemotherapy comprises treatment with an anthracycline agent.

5. The method of claim 1 comprising assessing both the HER2 locus and the TOP2A locus and the patient's likely response to a combination therapy comprising treatment with Herceptin® and an adjuvant chemotherapy.

6. A method for detecting a chromosomal rearrangement of a genetic locus X in a patient comprising:
(a) determining the copy number (x) of one or more segments of genomic DNA comprising a genetic locus X (X) relative to that of a linked chromosomal region (R) present in DNA extracted from one or more cancer cells of the patient;
(b) determining the copy number (i), relative to the copy number (r) of said region (R), of one or more segments of genomic DNA (I) interspersed between said genetic locus X and said centromere;
wherein if the copy number (i) of said interspersed region (I) differs from that of said region (R) or said genetic locus X (X), it is determined that there is a chromosomal rearrangement of the genetic locus (X) in the cancer cells of the patient.

7. The method of claim 1 or 6, wherein said genetic locus X is a HER2 locus.

8. The method of claim 7, wherein said linked chromosomal region (R) comprises a TOP2A locus, a RARA locus, or one or more other loci in q17-q21.2 of chromosome 17.

9. The method of claim 1 or 6, wherein said genetic locus X is a TOP2A locus.

10. The method of claim 9, wherein said linked chromosomal region (R) comprises a HER2 locus, a RARA locus, or one or more other loci in q17-q21.2 of chromosome 17.

11. The method of claim 1 or 6, wherein said linked chromosomal region (R) is a chromosomal centromere linked to said genetic locus X.

12. The method of claim 1 or 6, wherein the copy number (r) of said region (R) is higher than that of said genetic locus (X) and said interspersed region (I).

13. The method of claim 1 or 6, wherein the copy number (r) of said region (R) is higher than that of said interspersed region (I).

14. The method of claim 1 or 6, wherein the copy number (r) of said region (R) is higher than that of each of said genetic locus X (X) and interspersed region (I).

15. The method of claim 14, wherein the copy number (i) of said interspersed region (I) is higher than that of said genetic locus X (X).

16. The method of claim 1 or 6, wherein the copy number (r) of said region (R) is lower than that of said interspersed region (I).

17. The method of claim 1 or 6, wherein the copy number (r) of said region (R) is lower than that of each of said genetic locus X (X) and interspersed region (I).

18. The method of claim 17, wherein the copy number (i) of said interspersed region (i) is lower than that of said genetic locus X (X).

19. The method of claim 1 or 6, wherein the copy number (r) of said region (R) is about the same as that of said genetic locus X (X).

20. A probe for detecting a chromosomal rearrangement of a genetic locus X in a patient, wherein said probe hybridizes to one or more genomic segments (I) interspersed between said genetic locus X and a linked chromosomal region, said probe capable of determining whether the relative copy number (i) of said interspersed region (I) is lower than that of either or both of said linked region (R) and said genetic locus X (X).

21. The probe of claim 20, wherein said linked chromosomal region is a chromosomal centromere linked to said genetic locus (X).

22. The probe of claim 20, wherein said genetic locus X is selected from the group consisting of: HER2, TOP2A, and RARA.

23. The probe of claim 22, wherein said linked chromosomal region comprises one or more loci other than the genetic locus X in q17-q21.2 of chromosome 17.

24. A kit comprising the probe of claim 20.

25. A method for separately profiling genomic rearrangements at closely linked genetic loci (X) and (Y) in a single simultaneous experiment, comprising:
(a) determining the relative copy number of one or more segments of genomic DNA comprising a genetic locus (X) relative to that of a linked chromosomal region (R) present in DNA extracted from one or more cells of the patient; and
(b) determining the relative copy number of one or more segments of genomic DNA comprising a genetic locus (Y) relative to that of a linked chromosomal region (R) present in DNA extracted from one or more cells of the patient; wherein steps (a) and (b) are performed simultaneously in one experiment.

26. The method of claim 25, wherein said genetic loci (X) and (Y) are HER2 (H) and TOP2A (T).

27. The method of claim 25, wherein said linked chromosomal region is a linked chromosomal centromere.

28. The method of claim 25, wherein said linked chromosomal region comprises one or more other loci in q17-q21.2 of chromosome 17.
